SEQUENCE LISTING (29 sequences)

SEQ ID NO: 1 - DNA, 68 bp, Artificial Sequence
  Description of Artificial Sequence  Synthetic Primer
gcgttcggcaacactttctcttatggtgttcaagcacttgtcaagtgcttgaacaccataagagaaag

SEQ ID NO: 2 - DNA, 73 bp, Artificial Sequence
  Description of Artificial Sequence  Synthetic Primer
tgttcaagatcgggtctgcagatctccggatacnnnnnnnnnnnnnnnttctctttctacactggctagcaac

SEQ ID NO: 3 - DNA, 58 bp, Artificial Sequence
  Description of Artificial Sequence  Synthetic Primer
caagatggattgcacgcaggttctctgactgcaaccagtcggagaggcccacctctcc

SEQ ID NO: 4 - DNA, 62 bp, Artificial Sequence
  Description of Artificial Sequence  Synthetic Primer
cggccggagaacctgcgtgcaatccgtttcgtcggagcatcagtctattagtacgctttgct

SEQ ID NO: 5 - DNA, 15 bp, Artificial Sequence
  Description of Artificial Sequence  Synthetic Primer
ggagcatcagtctat

SEQ ID NO: 6 - DNA, 15 bp, Artificial Sequence
  Description of Artificial Sequence  Synthetic Primer
aaccagtcggagagg

SEQ ID NO: 7 - DNA, 15 bp, Artificial Sequence
  Description of Artificial Sequence  Synthetic Primer
cggagcatcagtcta

SEQ ID NO: 8 - DNA, 15 bp, Artificial Sequence
  Description of Artificial Sequence  Synthetic Primer
caaagtttggctccc

SEQ ID NO: 9 - DNA, 15 bp, Artificial Sequence
  Description of Artificial Sequence  Synthetic Primer
cacgtacgctgtcac

SEQ ID NO: 10 - DNA, 15 bp, Artificial Sequence
  Description of Artificial Sequence  Synthetic Primer
tggttggtaaaaatt

SEQ ID NO: 11 - DNA, 1303 bp, Homo sapiens
gtccaggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcgactcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 12 - DNA, 521 bp, Homo sapiens
ggacaaggctactataacaagcctgtggggcaaggtgaatgtggaagatgctggaggagaaaccctgggaaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaagctcctgggaaatgtgctggtgaccgttttggcaatccatttcggcaaagaattcacccctgaggtgcaggcttcctggcagaagatggcagaagatggtgactggagtggccagtgccctgtcctccagataccactgagctcactgcccatgatgcagagctttcaaggataggctttattctgcaagcaatacaaataataaatctattctgctaagagatcac

SEQ ID NO: 13 - DNA, 69 bp, Artificial Sequence
  Description of Artificial Sequence  Synthetic Primer
actcccaaagaatgcgtttcgctgtctccgattggaggagcatcagtctatccgaaggggaguaaggau

SEQ ID NO: 14 - DNA, 83 bp, Artificial Sequence
  Description of Artificial Sequence  Synthetic Primer
tggaagtactcccaaagaatgggattctttggaagcattcctacaaccagtcggagaggaaggactcgcctccggtcccatcc

SEQ ID NO: 15 - DNA, 83 bp, Artificial Sequence
  Description of Artificial Sequence  Synthetic Primer
tggaagtactcccaaagaatgggattctttggaagcattcctactggttggtaaaaattaaggactcgcctccggtcccatcc

SEQ ID NO: 16 - DNA, 19 bp, Artificial Sequence
  Description of Artificial Sequence  Synthetic Primer
ttgttggctgttcataacc

SEQ ID NO: 17 - DNA, 16 bp, Artificial Sequence
  Description of Artificial Sequence  Synthetic Primer
gccgtggctacttgct

SEQ ID NO: 18 - DNA, 20 bp, Artificial Sequence
  Description of Artificial Sequence  Synthetic Primer
ggtcagcttgccttaagtgg

SEQ ID NO: 19 - DNA, 21 bp, Artificial Sequence
  Description of Artificial Sequence  Synthetic Primer
ttttgttggctgttcataacc

SEQ ID NO: 20 - DNA, 13 bp, Artificial Sequence
  Description of Artificial Sequence  Synthetic Primer
ggcacgcggcgct

SEQ ID NO: 21 - DNA, 59 bp, Artificial Sequence
  Description of Artificial Sequence  Synthetic Primer
tctccacaggagtcacgtttcgtctatctgaccgncgguagaugguaaggaugggauau

SEQ ID NO: 22 - DNA, 73 bp, Artificial Sequence
  Description of Artificial Sequence  Synthetic Primer
tctccacaggagtcacgtttcgtctatctgaccgcggagcatcagtctacgguagaugguaaggaugggauau

SEQ ID NO: 23 - DNA, 56 bp, Artificial Sequence
  Description of Artificial Sequence  Synthetic Primer
actcccaaagaatgcgtttcgctgtctccgattggantccgaaggggaguaaggau

SEQ ID NO: 24 - DNA, 14 bp, Artificial Sequence
  Description of Artificial Sequence  Synthetic Primer
aagtggcatcgaag

SEQ ID NO: 25 - DNA, 20 bp, Artificial Sequence
  Description of Artificial Sequence  Synthetic Primer
gccaacaaaaggtcaggttg

SEQ ID NO: 26 - DNA, 36 bp, Artificial Sequence
  Description of Artificial Sequence  Synthetic Primer
ttttggtcagcttgccttaagtggcatcgaagtttt

SEQ ID NO: 27 - DNA, 30 bp, Artificial Sequence
  Description of Artificial Sequence  Synthetic Primer
cttacggcaagctgagcaagtagccacggc

SEQ ID NO: 28 - DNA, 68 bp, Artificial Sequence
  Description of Artificial Sequence  Synthetic Primer
gccacttaaggcaagctgaccttttgttggctgttcataacccantggggcgataaggtttcttcgat

SEQ ID NO: 29 - DNA, 56 bp, Artificial Sequence
  Description of Artificial Sequence  Synthetic Primer
cttacggctagctgaccgttggctgttcataangagggggagggggagggaggagg

(19) United States
(12) Patent Application Publication
(10) Pub. No.: US 2004/0063922 A1
(43) Pub. Date: Apr. 1, 2004

(54) Methods and compositions for catalytic DNA exchange in a sequence specific manner

(76) Inventor: Charles A. Conrad, Spring, TX (US)

Correspondence Address: FULBRIGHT & JAWORSKI, LLP, 1301 MCKINNEY, SUITE 5100, HOUSTON, TX, 77010-3095, US

(21) Appl. No.: 10/391,415

(22) Filed: Jul. 21, 2003

Related U.S. Application Data
(63) Continuation-in-part of application No. 09/836,608, filed on Apr. 17, 2001, now abandoned.
(63) Continuation-in-part of application No. 09/836,136, filed on Apr. 17, 2001, now abandoned.
(63) Continuation-in-part of application No. 09/836,358, filed on Apr. 17, 2001, now abandoned.
(63) Continuation-in-part of application No. 09/836,366, filed on Apr. 17, 2001, now abandoned.
(63) Continuation-in-part of application No. 09/836,126, filed on Apr. 17, 2001, now abandoned.

(51) Int. Cl. C07H 21/04; C12N 9/16; C12Q 1/68

(52) U.S. Cl. 536/23.2; 435/69.1; 435/196; 435/320.1; 435/325; 435/6

(57) ABSTRACT

Methods and compositions regarding autocatalytic DNA strand exchange with a pre-defined target DNA are described herein. Specifically, single stranded DNA or double stranded DNA construct comprising preferably four definable sequences facilitate phospho-diesterase hydrolysis reactions and subsequent phosphodiesterification reactions or a transesterification reaction between the construct and a target DNA. The reactions require no mediation by protein catalytic mechanisms and are useful for therapeutic applications regarding genetically related diseases.

The present invention claims priority to the following patent applications, all of which are incorporated by reference herein in their entirety: U.S. Nonprovisional patent application Ser. No. 09/836,136, filed Apr. 17, 2001; U.S. Nonprovisional patent application Ser. No. 09/836,358, filed Apr. 17, 2001; U.S. Nonprovisional patent application Ser. No. 09/836,608, filed Apr. 17, 2001; U.S. Nonprovisional patent application Ser. No. 09/836,366, filed Apr. 17, 2001; U.S. Nonprovisional patent application Ser. No. 09/836,126, filed Apr. 17, 2001; U.S. Provisional Patent Application No. 60/197,857, filed Apr. 14, 2000; U.S. Provisional Patent Application No. 60/197,860, filed Apr. 14, 2000; U.S. Provisional Patent Application No. 60/197,856, filed Apr. 14, 2000; U.S. Provisional Patent Application No. 60/197,859, filed Apr. 14, 2000; and U.S. Provisional Patent Application No. 60/197,858.

### FIELD OF THE INVENTION

The present invention generally relates to the fields of molecular biology and medicine. In specific embodiments, the present invention provides methods and compositions that allow for site-directed mutagenesis by specifically replacing a DNA sequence, and more particularly, to a single or double stranded nucleic acid construct each strand of which comprises preferably four components that collectively are capable of achieving autocatalytic DNA replacements, requiring no mediation by protein catalytic reactions. The invention further relates to methods and compositions for achieving this replacement with an outlook to effecting rehabilitation of a defective gene or to gene knockout in a site-specific manner in physiological or in vivo conditions.

### BACKGROUND OF THE INVENTION

Gene therapy encompasses a set of techniques that are designed to correct or replace a defective or harmful gene that leads to a particular disease state. Broadly speaking, gene therapy can target germ line tissues in which the correction would be carried forward with successive generations or it can target somatic cells in a particular tissue or tissues of interest, which will then not be carried forward to successive generations.

In the past, gene therapy has used an entire replacement gene therapy strategy in which the entire coding region of the gene plus exogenous controlling mechanisms such as promoters, enhancers and poly-adenylation signals are incorporated into the gene replacement strategy. This particular type of strategy is plagued with a number of problems including the large amount of genetic materials that need to be introduced into target cells, the need to insert the particular exogenous gene construct into the cellular genome, and significant delivery issues that will target the exogenous gene to the appropriate tissues.

Each of these problems can be substantial hurdles to overcome. As an example, in terms of the size of genes that need to be delivered, the gene encoding dystrophin, which is defective in patients with Duchenne muscular dystrophy, has been limited because of the size of this very large protein (Rando et al., 2000; Schwartz and Leterrier, 1999; Hartigan-O'Connor and Chamberlain, 2000). In terms of the controlling elements needed for gene therapy, virtually all gene products within the cell need to be turned on and off at appropriate times, depending on the environmental changes or the physiologic temperature requirements of the cell. This is not possible with exogenous gene constructs in this form of gene therapy, which loses all the natural regulatory mechanisms normally found in association with the natural or endogenous gene.

Lastly, delivery issues have been a major obstacle, and retrovirus or lentivirus (such as HIV-type virus) have been used to stably integrate the exogenous gene within the chromosomes. This type of integration, however, is a random event, and significant problems can occur if the exogenous gene is inserted in a critical area within a chromosome or if, perhaps, the regulatory enhancers or promoters insert next to, for instance, a potential oncogene.

A much more elegant system that alleviates virtually all of these problems would be a system that can correct individual nucleotide bases. This would account for the majority of genetic diseases based on single nucleotide mutations. This type of system, such as the DNA strand exchange system, allows for so-called “gene surgery” and negates the need to supply very large DNA constructs. Additionally, this type of system allows all of the natural regulatory mechanisms of the endogenous gene to be maintained. Furthermore, this type of system obviates the problem of random insertion of large genetic material randomly within chromosomal structure.

Thus, a means for selectively replacing fragments within a duplex DNA molecule in a site-specific manner, thereby obviating the need for gene replacement therapy, gene inhibition or “knock-out” techniques, has been a long-sought and desirable goal. Current methods of gene therapy have focused on replacing entire genes in order to over express a desired protein product which is dysfunctional in an organism due to genetic mutations. This entire replacement strategy has been attempted to effect the cure of such conditions as Sickle Cell Anemia (May et al., 2000; Xu et al., 2000), cystic fibrosis (Rubenstein et al., 1997; Hyde et al., 2000) or severe combined immuno-deficiency (SCID) (Fisher, 2000; Kohn, 2000), but these attempts are often problematic due to the complex regulatory mechanisms present in the organism that cannot be duplicated by current gene replacement approaches. Addressing these issues may be critical since most genes function in response to the needs for that particular gene produce according to the environmental circumstances of the organism. Among the most commonly occurring complications utilizing a gene replacement strategy, is “gene silencing” which effectively stops the expression of the newly-introduced gene within the cell. To overcome this problem, the organism is typically flooded with the replacement gene, leading to problems of increased toxicity. Moreover, size constraints associated with the transfer vector technology used in replacing the gene are often present.

It is known in the art that antisense strategies essentially down-regulate the messenger (mRNA), which is the first step required to produce active proteins or enzymes from a gene. The antisense strategies are numerous, including synthetic and synthetically made antisense oligonucleotides that can be modified in multiple ways to enhance their ability to bind to messenger mRNA or resist degradation. Additionally, the newly described interfering RNA (iRNA) is another technique for the down-regulation of messenger mRNA. However, all of these techniques essentially are focused on reduction of a harmful or unwanted gene product by the inhibition of the RNA component. Antisense strategies, however, cannot be used to perform a “gain of function” where a mutated and needed gene product is required to alleviate a particular diseased state. Additionally, antisense strategies are not permanent solutions; they require repeated dosing to down-regulate the production of harmful or unwanted massager RNA species.

A natural process of recombination occurs routinely between double stranded DNA in vivo between homologous strands catalyzed by complex cellular enzymatic machinery. Current in vitro recombinant DNA techniques, by which two double stranded DNA molecules may be spliced together, use exogenously produced enzymes, such as restriction endonuclease digestion and enzymatic ligation reactions. Sequence specific monofilament DNA exchange reactions have also been demonstrated in some in vivo DNA repair functions, such as the prototypic Rec A protein repair function in prokaryotic systems (Singer et al., 1999). These reactions involve an exchange of a monofilament homologous single stranded DNA molecule for the homologous portion of a double stranded target, the reaction being enzymatically promoted by Rec A protein. Recently, there has been a description of a single stranded DNA/RNA molecule that reportedly is able to correct a point mutation in vivo (Rando et al., 2000; Igoucheve and Yoon, 2000; Zhu et al., 1999; Kren et al., 1999; Kren et al., 1998). However, this reaction is very inefficient, effecting a correction of less than 1%, and the mechanism of action is completely unknown. Another site specific mechanism employs a triplex-forming oligonucleotide (TFO) structure single-stranded DNA to correct or change a double stranded DNA target, but this process is also very inefficient (about approximately 0.001 percent) (Vasquez et al., 2000). However, spontaneous exchange reactions between two double-stranded DNA molecules, without protein enzymatic assistance or manipulation, have not been previously described.

Thus, current methods of gene therapy would be significantly altered if it were possible to exchange DNA strands in a sequence specific manner and in a directed fashion. By repairing a defective gene “in situ” rather than by replacing the entire gene, all of the mechanisms that regulate its functioning would remain intact. Ideally, the repair would involve the replacement of from one to a few incorrect DNA base pairs in the defective natural gene sequence, thus preserving the natural regulatory mechanisms. This may be achieved through the exchange of duplex DNA strands in a sequence specific manner. The mechanism preferably would be applicable in vivo, would be sequence specific and would be relatively efficient. In another desirable aspect, DNA bases would be replaced to inactivate an over expressed gene such as an oncogene. By selectively repairing targeted base pairs of a DNA strand, the need for gene replacement therapy may be obviated.

In the embodiment wherein DNA bases are replaced to inactivate or disrupt an overexpressed gene, protein disruption would occur at the genetic level, garnering a tremendous therapeutic advantage since only the two alleles present in normal cells would be subjected to this approach. Current anti-sense therapies are targeted at the mRNA level wherein a thousand- to a million-fold amplification of this underlying gene sequence occurs. Worse still, at the level where most traditional pharmaceuticals work, protein amplification from the mRNA level is between 10- and 1000-fold. Thus, inhibiting a gene by this novel technique would theoretically translate into multiple-fold efficiencies over current techniques.

It has been known for some time that with the appropriate divalent cations as cofactors, such as ribozymes, RNA molecules can fold to assume configurations that will confer enzymatic function. Ribozymes were originally discovered in the early 1980's in nature and have been since modified extensively to function for therapeutic uses (Cech, 1992).

In 1994, an artificial selection scheme was used by researchers to isolate DNA sequences that were capable of cleaving RNA enzymatically (Breaker et al., 1994). Since that time, a number of DNA sequences have been discovered which confer many different enzymatic functions. Many of these enzymatic functions include, for example, RNase activity (Santoro, 1997; 1998; Breaker, 1994), copper dependent DNase activity (Carmi et al., 1998), copper-dependent DNA ligase activity (Cuenoud et al., 1995), calcium-dependent DNA kinase activity (Li et al., 1999). Except for the RNase enzymes that were discovered by using physiological conditions, these DNA enzymes do not appear to function under physiological conditions.

U.S. Pat. Nos. 5,565,350; 5,731,181; 5,760,012; 5,756,325; 5,795,972; 5,871,984; and 5,888,983 are directed to methods and compositions regarding an RNA/DNA chimera to perform nucleotide changes, particularly single nucleotide changes, utilizing host homologous recombination systems in a bacterial cell.

Cuenoud et al. (1995) and U.S. Pat. No. 5,910,408 are directed to catalytic DNA comprising ligase activity, and methods and compositions directed thereto.

U.S. Pat. Nos. 5,807,718; 6,110,462; and 6,326,174 relate to methods and compositions for catalytic DNA capable of cleaving RNA, and particularly relate to the endonuclease activity of a DNA for site-specific hydrolytic cleavage of a phosphoester bond.

U.S. Pat. No. 5,861,288 and WO 95/11304 regard cleavage of a bond between two ribonucleotides in an RNA-containing molecule, particularly by a catalytic DNA.

U.S. Pat. No. 6,159,714 is directed to a non-RNA containing enzymatic nucleic acid having endonuclease activity regarding cleavage of an RNA molecule.

Thus, in contrast to the known art, the present invention provides novel, useful, and efficient methods and compositions for DNA strand exchange in a sequence specific manner, particularly to a DNA molecule having both phosphodiesterase hydrolysis and phosphodiesterification functions.

### SUMMARY OF THE INVENTION

Whole gene replacement strategies (or also known as “gain of function”) carry with it a large number of problems, some of which are mentioned above. The term “whole gene replacement” is also somewhat misleading, since natural genes, which occur on the chromosomes, are actually broken-up into multiple small coding regions called exons. These exons are actually the information that eventually gives rise to the information to build a protein or enzyme. These exons are interrupted by long stretches of DNA called introns and are naturally snipped out during the processing of messenger RNA (mRNA). In gene replacement strategies, because the entire gene containing both introns and exons are so large (in the case of Duchenne muscular dystrophy, the abnormal protein dystrophin spans over 100,000 based-pairs in length) the size of genetic material is far too large to package and deliver into target cells. Therefore, a strategy has been developed to try to reduce the size of genetic material needed for whole gene replacement therapy. This technique relies on making a copy of the fully processed messenger RNA (mRNA). This is then called cDNA (which stands for a “copy” DNA). These cDNAs essentially represent all of the coding exons of a particular gene with the introns already snipped out. This process leads to an artificial exogenous gene product that is then connected to artificial promoters, in most cases, and other artificial signals that are required for the full processing of this cDNA back into RNA and eventually into a specific protein or enzyme.

This approach, however, is quite problematic, since we are now learning that many of the regulatory mechanisms for appropriate gene expression actually reside within these introns that are no longer present in this artificial exogenous gene construct. These problems, as well as those mentioned above, underscore dramatically the many difficulties that currently exist in gene replacement therapy strategies. A much more elegant and powerful system would be one in which repairs to the defective gene occur in situ and in a site-specific manner. This is sometimes referred to as “gene surgery” and absolutely represents the most preferred way to either repair a defective gene (gain of function) or alternately could be used to disrupt a gene in a site-specific manner (so-called loss of function strategies or down-regulation strategies). It is for this reason that the inventor specifically has developed a gene surgery system, which by its mechanism is far superior to gene replacement strategies for “gain of function” therapies and far superior to “loss of function” strategies such as currently employed by antisense approaches.

The present invention fulfills a need in the art by providing nucleic acid sequences having catalytic properties useful to perform DNA strand exchange reactions, thereby achieving tremendous simplification and efficiency of these reactions. Using a nucleic acid strand that can autonomously perform all of the reactions necessary to substitute a nucleic acid fragment with a target DNA substrate simplifies the reaction by eliminating the need for cumbersome additional enzymatic protein accompaniment.

The present invention provides methods involving single strand nucleic acid constructs having catalytic domains, sequence specific targeting domains and active phophorylated ends suitable for conducting reactions for the substitution of single strand nucleic acid sequences into target duplex DNA structures unilaterally. These methods allow for a different methodology in gene therapy by either repairing a defective genetic element or by introducing DNA and/or RNA base(s) to disrupt the overexpressed genetic elements. An example of a technique used in disrupting gene expression includes inserting nucleotide bases causing premature protein translation to stop (i.e. inserting a “stop” codon) prematurely. Inserting an “in frame” stop codon (e.g. TAA, TGA, or TGA) could be accomplished easily, although the insertion would need to be tailored in a sequence specific fashion for each gene “target” of interest. In another example, nucleotide bases would be inserted to disrupt normal transcriptional signals, such as promoter/enhancer signals. Signals involved in the beginning of normal translation such as Kozac consensus sequences or the initial “start” codon ATG could be disrupted as could normal splicing mechanisms. Normal poly-adenylation signals during transcription and immediate post-transcriptional events could be disrupted in a similar manner. Those skilled in the art will recognize a number of equivalent gene disruption events using various nucleotide base exchange reactions are achievable by the methods outlined herein.

In particular embodiments, the methods and compositions provided herein are utilized for applications other than therapeutic. For example, specific genetic changes may be made in a plant, animal, or even prokaryotic cell, such as a bacterial cell, so long as the target DNA is capable of being subject to a transesterification reaction (in some embodiments referred to as cleavage and ligation reactions). In some aspects of the invention, applications for agriculture, biomedical research, pharmaceutical production, and so forth utilize methods and/or compositions of the present invention. For agricultural purposes, a nucleic acid of a plant cell may be altered to comprise sequence(s) that renders one or more desirable characteristics to the cell, particularly when the cell is comprised in the plant. For example, the altered nucleic acid may render the plant more resistant to heat, cold, drought, pests, undesirable salinity, undesirable pH, and so forth.

The present invention particularly provides methods involving both unilateral and bilateral nucleic acid constructs having catalytic domains, sequence specific targeting domains and/or active phosphorylated ends suitable for ligation reactions, or a combination thereof. These methods allow for a different methodology in gene therapy by either fixing a defective genetic element or by introducing DNA and/or RNA base(s) to disrupt the over-expressed genetic elements.

The present disclosure improves known dissimilar concepts and can be viewed in simplistic terms of “gene surgery” on at least one particular DNA sequence(s) of interest.

The present invention overcomes the above-mentioned difficulties by providing a means suitable for accomplishing strand exchange with a target DNA substrate. According to the present invention, a single stranded monofilament or a double stranded nucleic acid comprising two monofilaments is provided that is capable of autocatalytically effecting a substitution of one or more nucleotide base pair(s) in order to correct genetic mutations or to disrupt a potentially harmful gene product. In a specific embodiment, the catalytic nucleic acid comprises no ribonucleotides, although in an alternative embodiment, the catalytic nucleic acid comprises at least one ribonucleotide, such as at an activated ligatable end.

In some embodiments of the present invention, there is a single stranded or double stranded nucleic acid including two strands, each of which are complementary to the other and each of which comprise, in preferred embodiments, four distinct sequence elements that may be tailored to improve reactivity with a designated substrate, although fewer of these elements may be utilized. The first of these sequences confers triplex forming oligonucleotide function to the target DNA substrate. The second sequence exhibits enzymatic characteristics capable of conducting both phosphodiesterase hydrolysis reactions (cleavage) and phosphodiesterfication reactions (ligation). The reactions may be sequential or simultaneous. Furthermore, the second sequence may utilize divalent cations, such as magnesium or others, as a physiological cofactor in the catalytic reactions.

The third sequence is essentially a homolog of the target DNA substrate comprising at least one nucleotide base difference with the target DNA, and may also contain some nucleotide base sequences that can also participate partially in triplex interactions to help stabilize the interactions between the target DNA sequence and the autocatalytic nucleic acid construct. In some embodiments, the nucleic acid is used to alter specifically a gene of interest by introducing into the gene nucleotides that can be different from the target DNA, can include nucleotides in addition to those present in the target DNA (an insertion), or can exclude nucleotides present in the target DNA (a deletion), or a combination thereof.

The fourth sequence comprises a terminal 5′ activated phosphor-imidazolide group, as well as terminal 5′ nucleotide bases. In specific embodiments, the two strands of the double stranded nucleic acid are annealed with one another to form a double strand structure wherein the two strands are bonded to one another according to the Watson and Crick rules throughout their respective sequences of complementary nucleotides.

In some embodiments, the double stranded nucleic acid is referred to as being comprised of two complementary monofilaments. In some embodiments, there is a monofilament nucleic acid sequence that includes the four distinct sequence elements described above, wherein the monofilament nucleic acid is capable of folding to mimic a double stranded nucleic acid. That is, the monofilament comprises appropriate sequence wherein Watson-Crick base pairing occurs intramolecularly. Furthermore, the monofilament nucleic acid is capable of autocatalyzing the exchange of the third sequence (homologous to the target DNA but comprising at least 1 nucleotide base difference compared to the target DNA substrate) with the target DNA substrate, causing both cleavage of the substrate as well as substitution and re-ligation of at least some of the third sequence (and comprising the at least one nucleotide difference between the two) with the target DNA sequence. Thus, the monofilament nucleic acid sequence of the present invention makes possible the replacement of single nucleotide aberrations as well as larger aberrations at physiological conditions, implying the possibility of performing “genetic surgery” at a genetic level in vivo.

Accordingly, it is an object of the present invention to use a monofilament “Active-Exchangeable single stranded or double stranded DNA” (AE-DNA) reaction to replace or exchange (in vivo and/or in vitro) at least one nucleotide base in a sequence specific manner of a single stranded or preferably double stranded target DNA of interest. However, the term AE-DNA may also refer to a molecule comprising a ribonucleotide, such as, for example, an RNA/DNA hybrid molecule or a RNA/DNA chimeric molecule. The target DNA may be a DNA molecule comprised of two monofilament strands or it may be comprised of one monofilament strand comprising a duplex DNA region. Another object of the present invention is to use a sequence of nucleotides having catalytic properties to perform unilateral AE-DNA strand exchange reactions.

In other embodiments, the double stranded nucleic acid construct is capable of autocatalyzing the exchange of the third sequence (described above) with the target DNA substrate, causing both cleavage of at least some of the third sequence (and comprising the at least one nucleotide difference between the two) with the target DNA sequence. The double stranded nucleic acid sequence of the present invention makes possible the replacement of single nucleotide base pair aberrations as well as larger aberrations at physiological conditions, implying the possibility of carrying out “genetic surgery” at a genetic level in vivo.

Another object of the present invention is to use a sequence of nucleotides having catalytic properties to perform unilateral and/or bilateral AE-DNA strand exchange reactions. Yet another object of the present invention is to use these composite nucleic acid sequences of interest to coordinate diverse double strand DNA exchange reactions in order to efficiently correct in vivo and/or in vitro genetic mutations and/or to efficiently disrupt the expression of a deleterious gene or abnormally functioning gene, and to do so efficiently.

The present invention is in contrast to some methods known in the art in that it does not require and is not dependent upon a machinery of a host cell, particularly given that the invention works both in vivo and in vitro.

In one embodiment of the present invention, there is a nucleic acid construct (“AE-DNA”) having at least one monofilament strand of nucleic acid comprising at least two nucleotide sequences, and a target DNA sequence, the at least two nucleotide sequences being the same as or different from one another, said at least two nucleotide sequences being selected from the group comprising a first sequence having a 3′ end and a 5′ end and being capable of forming triplex forming oligonucleotide interactions with a target DNA sequence according to Hoogsteen binding rules; a second sequence having a 3′ end and a 5′ end and being capable of catalyzing a sequence specific cleavage reaction through the hydrolysis of a phosphodiester linkage in the target DNA sequence; a third sequence having a 3′ end and a 5′ end and being homologous with the target DNA sequence; and a fourth sequence having a 3′ end and a 5′ end, wherein the 5′ end comprises an activated phosphor-imidazolide group, wherein the at least two nucleotide sequences being covalently bonded to one another by one of two fashions: 1) wherein said 3′ end of one nucleotide sequence is covalently bonded to said 5′ end of another nucleotide or 2) wherein the nucleotides of one sequence are pair bonded in a complementary fashion.

In a specific embodiment, at least one monofilament nucleic acid strand comprises few than four of said nucleotide sequences. The AE-DNA may comprise, in some embodiments, first and second monofilament nucleic acid strands, said first monofilament nucleic acid strand being bonded to said second monofilament nucleic acid strand by base pairing between said nucleotides of said first monofilament nucleic acid strand and said nucleotides of said second monofilament nucleic acid strand, and wherein at least one of the first and the second monofilament nucleic acid strands comprises fewer than four of the nucleotide sequences, hereinafter referred to as “semi-bilateral AE-DNA.”

In another specific embodiment, the AE-DNA comprises a first and a second monofilament DNA strands, the first monofilament nucleic acid strand being bonded to the second monofilament DNA strand by base pairing between the nucleotides of the first monofilament DNA strand and the nucleotides of the second monofilament nucleic acid strand, and wherein each of the first and the second monofilament DNA strands comprises fewer than four of the nucleotide sequences, such as wherein at least one of the first and the second monofilament nucleic acid strands does not comprise the first nucleotide sequence or the fourth nucleotide sequence (semi-bilateral AE-DNA”).

In the present invention, the AE-DNA may also comprise a first and a second monofilament nucleic acid strands, wherein the first monofilament nucleic acid strand is bonded to the second monofilament nucleic acid strand by base pairing between the nucleotides of the first monofilament nucleic acid strand and the nucleotides of the second monofilament nucleic acid strand, and wherein one of the first and the second monofilament nucleic acid strands is capable of completing a nucleic acid exchange reaction with the target DNA sequence. In an additional specific embodiment, at least one of the first and the second monofilament nucleic acid strands of the AE-DNA does not comprise the second nucleotide sequence or the first nucleotide sequence or the third nucleotide sequence.

In an additional specific embodiment, the AE-DNA comprises a first and a second monofilament nucleic acid strands, wherein the first monofilament nucleic acid strand is bonded to the second monofilament nucleic acid strand by base pairing between the nucleotides of the first monofilament nucleic acid strand and the nucleotides of the second monofilament nucleic acid strand, and wherein each of the nucleotide sequences of a respective first or second monofilament nucleic acid strand are covalently bonded to one another at respective 3′ ends and/or 5′ ends in either a 3′ end to 5′ end direction or a 5′ end to 3′ end direction.

In a further specific embodiment, both of the first and the second monofilament nucleic acid strands comprise at least one each of the first, second, third and fourth nucleotide sequences, although the strands may comprise a subset thereof. In a further specific embodiment, the first, second, third and fourth nucleotide sequences occur in each of the first or second monofilament nucleic acid strands in any order.

In another specific embodiment, at least one of the first and the second monofilament nucleic acid sequence contains the second nucleotide sequence, and wherein the first and said second monofilament nucleic acid sequences collectively or separately further comprise sufficient nucleotides selected from the group consisting of the first, said second, the third and the fourth nucleotide sequences to render the AE-DNA capable of completing an exchange of nucleotides with the target DNA sequence.

In an additional specific embodiment, one of the first and the second monofilament nucleic acid sequences contains a free 5′ terminal hydroxyl group and wherein the other of the first and the second monofilament sequences contains a 3′ terminal activated phosphor-imidazolide group. In a further specific embodiment, cleavage reaction of linking nucleotide basis catalyzed by the second sequence occurs between a purine and purine pair of the target DNA sequence, between a pyrimidine and pyrimidine pair of the target DNA sequence, or between a purine and pyrimidine pair of the target DNA sequence. In another specific embodiment, both of the first and the second monofilament nucleic acid sequences contains a free 5′ terminal hydroxyl group and a 3′ terminal activated phosphorimidazolide group.

In an additional embodiment of the present invention, there is a monofilament nucleic acid sequence, the sequence comprising a first sequence capable of forming triplex forming oligonucleotide interactions with a target DNA sequence according to Hoogsteen binding rules. In a specific embodiment, the monofilament further comprises a second sequence, capable of catalyzing a sequence specific cleavage reaction through the hydrolysis of a phosphodiester linkage in the target DNA sequence. In an additional specific embodiment, the phosphodiesterase hydrolysis reaction occurs between a DNA sequence containing a purine base and pyrimidine base junction, between a purine base and purine base junction, or between a pyrimidine base and pyrimidine base junction.

In another specific embodiment, the second sequence also is capable of catalyzing ligation reactions in a sequence specific manner to form ligative bonds between a first and a second strand of nucleic acid by formation of a phosphodiester covalent bond between two nucleotide bases, each of the first and second strands of nucleic acid having a 3′ end and a 5′ end. In an additional specific embodiment, each of the first and the second strands of nucleic acid have a free 3′ hydroxyl group and a 5′ activated phosphor-imadazolide group. In a further specific embodiment, the first nucleic acid strand contains a free 3′ hydroxyl group and wherein the second nucleic acid strand contains an activated phosphorimidazolide group at the 5′ end, and wherein the two nucleotide bases of the phosphodiester covalent bond are a purine and pyrimidine pair, a purine and purine pair, or a pyrimidine and pyrimidine pair.

In a specific embodiment, the monofilament further comprises a third sequence that is homologous with the target DNA sequence for up to about 100,000 DNA nucleotide bases and/or a fourth sequence having a 3′ end and a 5′ end, wherein the 5′ end comprises an activated phosphor-imidazolide group. In a specific embodiment, the fourth sequence has a 3′ end and a 5′ end and contains preferably, for example, up to about 10,000 nucleotide bases, in some embodiments, although the size may be greater, the fourth sequence being homologous to the DNA target sequence. In an additional specific embodiment, the fourth sequence comprises a 3′ end and a 5′ end and contains up to about 10,000 nucleotide bases, the fourth sequence being relatively homologous to the DNA target sequence, wherein at least one of the nucleotide bases differs from a corresponding base in the DNA target sequence.

In a first specific embodiment, the first, second, third and fourth sequences (collectively referred to as “AE-DNA”) react unilaterally and without the addition of a catalyst with a DNA target sequence, substituting the target sequence with the homologous fourth sequence through autocatalyzed cleavage and ligation reactions. In another specific embodiment, the first, second, third and fourth sequences (collectively referred to as “AE-DNA”) react unilaterally and without the addition of a catalyst with a DNA target sequence, substituting the target DNA sequence with the homologous third sequence through autocatalyzed cleavage and ligation reactions.

In an additional specific embodiment, the target DNA sequence is a predefined monofilament sequence capable of chemical reactions with the “AE-DNA”. In another specific embodiment, the target DNA sequence is capable of assuming a folded configuration in a complementary and anti-parallel fashion to mimic, at least in part, a double stranded nucleic acid molecule. In a further specific embodiment, the AE-DNA strand is capable of reacting under physiological conditions. In another specific embodiment, the second sequence may utilize magnesium divalent cations as a physiological cofactor in the catalytic reactions, although other cations may be utilized. Histidine may also be utilized in a reaction described herein.

In a specific embodiment, the enzymatic domain sequence has a 5′ end and a 3′ end and may be identified by the sequence: 5′-TGG TTG GTA AAA ATT-3′ (SEQ ID NO:10), wherein “T” referred to herein symbolizes a Thymine nucleotide, “G” referred to herein symbolizes a Guanine nucleotide and “A” referred to herein symbolizes an Adenine nucleotide. In an additional specific embodiment, the third sequence has a 5′ end and a 3′ end and may be identified by the sequence: 5′-AAC CAG TCG GAG AGG-3′ (SEQ ID NO:6), wherein “C” referred to herein symbolizes a Cytosine nucleotide. In another specific embodiment, the enzymatic domain sequence has a 5′ end and a 3′ end and may be identified by the sequence: 5′-CGG AGC ATC AGT CTA-3′ (SEQ ID NO:7); 5′-GGA GCA TCA GTC TAT-3′ (SEQ ID NO:5); 5′-CAA AGT TTG GCT CCC-3′ (SEQ ID NO:8); 5′-CAC GTA CGC TGT CAC-3′ (SEQ ID NO:9); or 5′-GGC ACG CGG CGC T-3′. (SEQ ID NO:20). These symbols are also utilized elsewhere herein.

In an additional specific embodiment, the second sequence is capable of catalyzing the sequence specific cleavage reaction and the ligation reactions in both in vivo and in vitro environments. In another specific embodiment, the monofilament DNA sequence may be delivered into in vivo systems by current conventional delivery techniques.

In another embodiment of the present invention, there is a method for treating an organism suffering from genetic point mutations in a target DNA sequence comprising the step of administering a therapeutically effective amount of a monofilament nucleic acid sequence to said organism, said DNA sequence being characterized as comprising: a) a first sequence capable of forming triplex forming oligonucleotide interactions with a target DNA sequence according to Hoogsteen binding rules; b) a second sequence capable of catalyzing a sequence specific cleavage reaction through the hydrolysis of a phosphodiester linkage in the target DNA sequence; c) a third sequence, the third sequence being homologous with the target DNA sequence; and d) a fourth sequence having a 3′ end and a 5′ end, wherein the 5′ end comprises an activated phosphor-imidazolide group.

In an additional embodiment of the present invention, there is a method for treating an organism suffering from errors in splicing mechanisms of a target DNA sequence comprising the step of administering a therapeutically effective amount of a monofilament nucleic acid sequence to the organism, the DNA sequence being characterized as comprising: a) a first sequence capable of forming triplex forming oligonucleotide interactions with a target DNA sequence according to Hoogsteen binding rules; b) a second sequence capable of catalyzing a sequence specific cleavage reaction through the hydrolysis of a phosphodiester linkage in the target DNA sequence; c) a third sequence, the third sequence being homologous with the target DNA sequence; and d) a fourth sequence having a 3′ end and a 5′ end, wherein the 5′ end comprises an activated phosphor-imidazolide group.

In a further embodiment of the present invention, there is a method for treating an organism by disrupting a gene function through the exchange of nucleotide bases to form “in frame” stop codons in a target DNA sequence comprising the step of administering a therapeutically effective amount of a monofilament nucleic acid sequence to the organism, the nucleic acid sequence being characterized as comprising: a) a first sequence capable of forming triplex forming oligonucleotide interactions with a target DNA sequence according to Hoogsteen binding rules; b) a second sequence capable of catalyzing a sequence specific cleavage reaction through the hydrolysis of a phosphodiester linkage in the target DNA sequence; c) a third sequence, the third sequence being homologous with the target DNA sequence; and d) a fourth sequence having a 3′ end and a 5′ end, wherein the 5′ end comprises an activated phosphor-imidazolide group.

In another embodiment of the present invention, there is a method for treating an organism by disrupting a gene function through the exchange of nucleotide bases to alter transcriptional start processes in a target DNA sequence comprising the step of administering a therapeutically effective amount of a monofilament nucleic acid sequence to the organism, the nucleic acid sequence being characterized as comprising: a) a first sequence capable of forming triplex forming oligonucleotide interactions with a target DNA sequence according to Hoogsteen binding rules; b) a second sequence capable of catalyzing a sequence specific cleavage reaction through the hydrolysis of a phosphodiester linkage in the target DNA sequence; c) a third sequence, the third sequence being homologous with the target DNA sequence; and d) a fourth sequence having a 3′ end and a 5′ end, wherein the 5′ end comprises an activated phosphor-imidazolide group.

In an additional embodiment of the present invention, there is a method for treating an organism by disrupting a gene function through the exchange of nucleotide bases in order to change post-transcriptional signaling in a target DNA sequence comprising the step of administering a therapeutically effective amount of a monofilament nucleic acid sequence to the organism, the nucleic acid sequence being characterized as comprising: a) a first sequence capable of forming triplex forming oligonucleotide interactions with a target DNA sequence according to Hoogsteen binding rules; b) a second sequence capable of catalyzing a sequence specific cleavage reaction through the hydrolysis of a phosphodiester linkage in the target DNA sequence; c) a third sequence, the third sequence being homologous with the target DNA sequence; and d) a fourth sequence having a 3′ end and a 5′ end, wherein the 5′ end comprises an activated phosphor-imidazolide group.

In another embodiment of the present invention, there is a nucleotide sequence, the sequence exhibiting catalytic properties under physiological conditions and having a cofactor comprising a divalent cation, such as magnesium, although other divalent cations and even monovalent cations, or mixtures thereof, may be utilized in some embodiments. In a specific embodiment, the nucleotide sequence comprises about 15 nucleotides bonded covalently to one another in a linear fashion, although other sizes may be utilized. In an additional specific embodiment, the nucleotide sequence comprises about 15 nucleotides bonded covalently to one another in a linear fashion.

In a further specific embodiment, the nucleotide sequence catalyzes the action of cleaving a DNA molecule. In an additional specific embodiment, the nucleotide sequence catalyzes the action of ligating two DNA molecules together. In a specific embodiment, the nucleotide sequence catalyzes the action of cleaving a first DNA molecule and of ligating the cleaved first DNA molecule to a second DNA molecule or a second DNA/RNA hybrid molecule. In a specific embodiment, the catalytic nucleotide sequence may be 5′-GGA GCA TCA GTC TAT-3′ (SEQ ID NO:5) (wherein “5′” and “3′” refer to the respective 5′ and 3′ ends of the nucleotide sequence); 5′-AAC CAG TCG GAG AGG-3′ (SEQ ID NO:6); 5′-CGG AGC ATC AGT CTA-3′ (SEQ ID NO:7); 5′-CAA AGT TTG GCT CCC-3′ (SEQ ID NO:8); 5′-CAC GTA CGC TGT CAC-3′ (SEQ ID NO:9); 5′-TGG TTG GTA AAA ATT-3′ (SEQ ID NO:10); or 5′-GGC ACG CGG CGC T-3′ (SEQ ID NO:20).

In a further specific embodiment, the nucleotide sequence may be incorporated into a larger nucleotide construct, the larger nucleotide construct comprising in addition to the nucleotide sequence (“first sequence”): a) a second sequence having a 3′ end and a 5′ end and being capable of forming triplex forming oligonucleotide interactions with a target DNA sequence according to Hoogsteen binding rules; b) a third sequence having a 3′ end and 5′ end and being homologous with the target DNA sequence; and c) a fourth sequence having a 3′ end and a 5′ end, wherein the 5′ end comprises an activated phosphor-imidazolide group.

In an additional embodiment of the present invention, there is a nucleotide sequence comprising a plurality of individual nucleotides, wherein each of the individual nucleotides are bonded one to another by covalent bonds to form a single strand, the single strand having a 3′ end and a 5′ end; and an activated phosphate-containing group, the activated phosphate-containing group comprising a phosphate group and an activating group and the activated phosphate-containing group being bound to either of the 3′ end or the 5′ end of the single strand. In a specific embodiment, the activating group is an imidazolide, such as one selected from the group consisting of 4,5-imidazoledicarboxylic acid and a 2-imidazolidonethione. In another specific embodiment, the activating group is an activating nucleotide, such as an adenine, thymine, guanine or a cytosine. In a further specific embodiment, the activating nucleotide is selected from the group consisting of uracil and inositol.

In another embodiment of the present invention, there is a nucleotide sequence that may be incorporated into a larger nucleotide construct, the larger nucleotide construct comprising in addition to the nucleotide sequence (“first sequence”): a) a second sequence having a 3′ end and a 5′ end and being capable of forming triplex forming oligonucleotide interactions with a target DNA sequence according to Hoogsteen binding rules; b) a third sequence capable of catalyzing a sequence specific cleavage reaction through the hydrolysis of a phosphodiester linkage in the target DNA sequence; and c) a fourth sequence having a 3′ end and 5′ end and being homologous with the target DNA sequence.

In an additional embodiment of the present invention, there is a nucleotide sequence comprising a plurality of individual nucleotides, wherein each of the individual nucleotides are bonded one to another by covalent bonds to form a single strand, the single strand having a 3′ end and a 5′ end; and an activated phosphate-containing group, the activated phosphate-containing group comprising a triphosphate group and the activated phosphate-containing group being bonded to either of the 3′ end or the 5′ end of the single strand. In a specific embodiment, the nucleotide sequence may be incorporated into a larger nucleotide construct, the larger nucleotide construct comprising in addition to the nucleotide sequence (“first sequence”) a) a second sequence having a 3′ end and a 5′ end and being capable of forming triplex forming oligonucleotide interactions with a target DNA sequence according to Hoogsteen binding rules; b) a third sequence capable of catalyzing a sequence specific cleavage reaction through the hydrolysis of a phosphodiester linkage in the target DNA sequence; and c) a fourth sequence having a 3′ end and 5′ end and being homologous with the target DNA sequence.

In another embodiment of the present invention, there is a double stranded nucleic acid construct (“AE-DNA”) having a first and a second monofilament DNA sequence, each of the monofilament DNA sequences comprising a first sequence capable of forming triplex forming oligonucleotide interactions with a target DNA sequence according to Hoogsteen binding rules. In a specific embodiment, each of the monofilament DNA sequences further comprises a second sequence capable of catalyzing a sequence specific cleavage reaction through the hydrolysis of a phosphodiester linkage in the target DNA sequence. In another specific embodiment, the phosphodiesterase hydrolysis reaction occurs between a DNA sequence containing a purine base and pyrimidine base junction or a pyrimidine base and pyrimidine base junction. In an additional specific embodiment, the second sequence also is capable of catalyzing ligation reactions in a sequence specific manner to form ligative bonds between a first and a second strand of DNA (or DNA/RNA hybrid) by formation of a phosphodiester covalent bond between two nucleotide bases, each of the first and second strands of DNA having a 3′ end and a 5′ end.

In a specific embodiment, each of the first and second strands of nucleic acid have a free 3′ hydroxyl group and a 5′ activated phosphor-imidazolide group. In an additional specific embodiment, the first nucleic acid strand contains a free 3′ hydroxyl group and wherein the second nucleic acid strand contains an activated phosphor-imidazolide group at the 5′ end, and wherein the two nucleotide bases of the phosphodiester covalent bond are a purine and pyrimidine pair, a purine and purine pair, or a pyrimidine and pyrimidine pair.

In another specific embodiment, each of the monofilament nucleic acid sequences further comprises a third sequence that is homologous with the target DNA sequence for up to, preferably and for example, about 100,000 DNA nucleotide bases. In an additional specific embodiment, each of the monofilament nucleic acid sequences further comprises a fourth sequence having a 3′ end and a 5′ end, wherein the 5′ end comprises an activated phosphor-imidazolide group. In another specific embodiment, each of the monofilament nucleic acid sequences further comprises a fourth sequence having a 3′ end and containing up to about 10,000 nucleotide bases, the fourth sequence being homologous to the DNA target sequence. In another specific embodiment, each of the monofilament nucleic acid sequences further comprises a fourth sequence having a 3′ end and containing up to, preferably and for example, about 10,000 nucleotide bases, the fourth sequence being relatively homologous to the DNA target, wherein at least one of the nucleotide bases differs from a corresponding base in the DNA target sequence.

In an additional specific embodiment, the first, second, third and fourth sequences of each of the monofilament nucleic acid sequences react without the addition of a catalyst with a DNA target sequence, substituting the target DNA sequence with the homologous fourth sequence through autocatalyzed cleavage and ligation reactions. In a further specific embodiment, the first, second, third and fourth sequences of each of the monofilament nucleic acid sequences react without the addition of a catalyst with a DNA target sequence, substituting the target DNA sequence with the homologous fourth sequence through autocatalyzed cleavage and ligation reactions. In an additional specific embodiment, the target DNA sequence is a predefined doubled stranded sequence capable of chemical reactions with the AE-DNA. In another specific embodiment, the AE-DNA strand is capable of reacting under physiological conditions. In an additional specific embodiment, the target DNA sequence is a gene sequence of an organism, such as a human. In another specific embodiment, the second sequences of interest are capable of utilizing, for example, magnesium divalent cations as a physiological cofactor in the catalytic reactions.

In a specific embodiment, the first monofilament nucleic acid sequence has a 5′ end and a 3′ end and may comprise the sequence 5′-(P-Im)-CGG CCG GAG AAC CTG CGT GCA ATC CGT TTC GTC GGA GCA TCA GTC TAT TAG TAC GCT TTG CT-3′ (SEQ ID NO:4), wherein “P-Im” symbolizes an activated phospho-imidazolide group. In another specific embodiment, the first monofilament nucleic acid sequence has a 5′ end and a 3′ end and may be comprise the sequence 5′-(P-Im)-CAA GAT GGA TTG CAC GCA GGT TCT CTG ACT GCA ACC AGT CGG AGA GGC CCA CCT CTC C-3′ (SEQ ID NO:3), wherein “P” symbolizes an activated phosphate group and “Im” symbolizes an imidazolide group. In a specific embodiment, the second sequence of the first and the second monofilament nucleic acid sequences is capable of catalyzing the sequence specific cleavage reaction and the ligation reactions in both in vitro and in vivo environments. In a further specific embodiment, the AE-DNA may be delivered intact into an in vivo system.

In another embodiment of the present invention, there is a method for treating an organism suffering from a condition having a genetic origin, comprising the step of administering a therapeutically effective amount of a double stranded nucleic acid construct having a first and a second monofilament nucleic acid sequences, each of the monofilament nucleic acid sequences comprising at least one of a) a first sequence capable of forming triplex forming oligonucleotide interactions with a target DNA sequence according to Hoogsteen binding rules; b) a second sequence capable of catalyzing a sequence specific cleavage reaction through the hydrolysis of a phosphodiester linkage in the target DNA sequence; c) a third sequence, the third sequence being homologous with the target DNA sequence; and d) a fourth sequence having a 3′ end and a 5′ end, wherein the 5′ end comprises an activated phosphor-imidazolide group. In a specific embodiment, the condition is derived from errors in splicing mechanisms and/or is remediable by forming a stop codon in a gene to disrupt a gene function and/or is remediable by altering the nucleotide base sequence of a gene to disrupt a transcriptional start process. In an additional specific embodiment, the start process is the start codon ATG. In a specific embodiment, the condition is remediable by altering the nucleotide base sequence of a gene to disrupt promoter/enhancer sequences and/or is remediable by altering the nucleotide base sequence of a gene to disrupt post-transcriptional signaling or post-translational signaling.

In one embodiment of the present invention, there is a nucleic acid comprising catalytic activity, the nucleic acid having a 5′ end and a 3 end′ and the catalytic activity defined as phosphodiesterase hydrolysis activity and phosphodiesterification activity.

In another embodiment of the present invention, there is a nucleic acid comprising catalytic activity, the nucleic acid having a 5′ end and a 3 end′ and the catalytic activity defined as transesterification activity.

In an additional embodiment there is a single-stranded autocatalytic nucleic acid molecule comprising transesterification activity. The molecule may be comprised of deoxyribonucleotides, ribonucleotides, or a combination thereof. In some embodiments, the molecule is further defined as comprising activity for replacing at least a portion of one strand of a double stranded molecule with at least a portion of said nucleic acid molecule.

The catalytic domain of the compositions described herein may, in some embodiments, comprise a consensus primary structure, a consensus secondary structure, a consensus tertiary structure, or a combination thereof.

In an embodiment of the present invention, there is a nucleic acid molecule having at least one enzymatic domain that provides both phosphodiesterase hydrolysis and phosphodiesterification functions, wherein the domain is obtainable by a process comprising a) identifying a target DNA molecule having a known target sequence; b) obtaining a tester nucleic acid molecule for testing for the desired enzymatic activity, such as, for example, the phosphodiesterase hydrolysis and phosphodiesterification activities; c) assaying whether at least a part of the tester nucleic acid molecule facilitates insertion of a sequence into the target sequence; and d) preparing the DNA molecule having the enzymatic domain by producing the molecule comprising the enzymatic activity sequence identified in the tester.

The assaying step may be further defined as providing a double stranded target DNA region, the target region defined as an acceptor region; providing a single stranded donor molecule; providing the tester molecule; and assaying for action of the donor molecule upon said acceptor region. The assaying for action of the donor molecule upon the acceptor region may further be defined as assaying for replacement of at least a part of the acceptor region with at least a part of the donor molecule. In some embodiments, the tester molecule and the donor molecule may be the same molecule. At least a portion of the tester molecule may further be defined as comprising a folded complementary anti-parallel configuration.

In particular aspects of the invention, the double stranded DNA region is comprised of two monofilament molecules and one of the monofilament molecules is the tester molecule.

In other aspects, the phosphodiesterase hydrolysis and phosphodiesterification functions occur in a one-step process.

The tester molecule may further be defined as comprising one or more of the following an activated ligatable end; an enzymatic domain, wherein said domain comprises the phosphodiesterase hydrolysis and phosphodiesterification functions; and a DNA sequence homologous to the target DNA region. The tester molecule may also further comprise a triplex forming oligonucleotide domain. In a specific embodiment, the activated ligatable end is located at the 5′ end of the DNA or is located at the 3′ end of the DNA. The activated ligatable end can comprise an activating group and/or a phosphate group. The activating group can be an imidazolide, in some embodiments, such as, for example, 4,5-imidazoledicarboxylic acid or 2-imidazolidonethione.

The nucleic acid molecules of the present invention may further comprise a phosphate group source, such as, for example, a nucleotide such as, for example, adenosine triphosphate. The activated ligatable end may comprise a phosphor-imidazolide group, and/or the activated ligatable end comprises adenine deoxyribonucleoside, guanine deoxyribonucleoside, thymine deoxyribonucleoside, cytosine deoxyribonucleoside, inositol ribonucleoside, or uracil ribonucleoside, in some embodiments.

Enzymatic domains described herein comprise SEQ ID NO:5, SEQ ID NO:6, SEQ ID NO:7, SEQ ID NO:8, SEQ ID NO:9, SEQ ID NO:10, SEQ ID NO:20, or combinations or mixtures thereof.

In specific embodiments, the target DNA region comprises human genomic sequence. The DNA sequence homologous to the target DNA region is further defined as comprising a therapeutic alteration compared to the target DNA region, in some embodiments.

The nucleic acid molecules of the present invention may be comprised in a composition that further comprises a divalent cation, such as Ba²⁺, Sr²⁺, Ca²⁺, Mg²⁺, Mn²⁺, Fe²⁺, Co²⁺, Ni²⁺, Cu²⁺, Zn²⁺, Pb²+, Cd²⁺, or a mixture thereof.

The tester molecule may further be defined as comprising the enzymatic domain, wherein the domain comprises the phosphodiesterase hydrolysis and phosphodiesterification functions. In some embodiments, the tester molecule is a closed circular molecule.

The assay step for action of the donor molecule upon the acceptor region comprises polymerase chain reaction, in some embodiments.

In another embodiment of the present invention, there is a method of exchanging a nucleic acid sequence of interest with a target DNA region, comprising providing a nucleic acid molecule as described herein, wherein the sequence inserted into the target sequence is referred to as the nucleic acid sequence of interest; providing the target DNA region; and introducing the nucleic acid sequence of interest to the target DNA region, wherein the phosphodiesterase hydrolysis and phosphodiesterification functions of the nucleic acid molecule from claim 1 exchanges at least a portion of the nucleic acid sequence of interest with the target DNA region. The nucleic acid molecule as described herein may further be defined as comprising at least one of the following: an activated ligatable end; the enzymatic domain, the domain comprising the phosphodiesterase hydrolysis and phosphodiesterification functions; and the nucleic acid sequence of interest, wherein the sequence is homologous to the target DNA region, wherein there is at least one nonidentical base pair between the nucleic acid sequence of interest and the target DNA region. In a specific embodiment, the nucleic acid molecule further comprises a triplex forming oligonucleotide domain.

In some embodiments of the invention, the phosphodiesterase hydrolysis and/or said phosphodiesterification functions further comprise the use of histidine, of a divalent cation, or of both. The method occurs under physiological conditions, in some embodiments, and it may occur either in vitro, in vivo, or ex vivo. In specific embodiments, the method occurs in a cell, such as, for example, a cell in a human afflicted with a disease of genetic origin, the disease the indirect or direct result of a defect in the target DNA region. In other embodiments, the method occurs in a plant cell, such as, for example, a plant cell comprised in a plant.

In an additional embodiment of the present invention, there is a method of treating an individual afflicted with a disease of genetic origin, the disease of genetic origin comprising a defective DNA sequence, comprising the step of exchanging a nondefective DNA sequence with the defective DNA sequence using a DNA prepared as described herein. The defect in the DNA sequence may be a point mutation, an inversion, a deletion, a frameshift mutation, or a combination thereof. The defect in the DNA sequence may also comprise an error in a splicing mechanism or an error in a regulatory mechanism.

In another embodiment, there is a method of treating an individual afflicted with a disease related to an undesirable gene product by affecting the gene product level or activity in a cell of the individual, the method comprising the step of exchanging a first DNA sequence with a second DNA sequence using a DNA prepared as described herein, and wherein the exchanging step results in the affecting of the gene product level or activity. The affecting of the gene product level or activity comprises, in specific embodiments, introducing a stop codon into nucleotide sequence that encodes the undesirable gene product; reducing the transcriptional level or rate of the undesirable gene product; altering post-transcriptional processing of the undesirable gene product; or a combination thereof. In specific embodiments, the catalysis function for the phosphodiesterase hydrolysis and phosphodiesterification reactions is provided by a DNA molecule comprising the first DNA sequence.

In another embodiment of the present invention, there is a method of identifying, or isolating, a nucleic acid molecule comprising at least one enzymatic domain that provides both phosphodiesterase hydrolysis and phosphodiesterification functions, comprising: a) identifying a target DNA molecule having a known target sequence; b) obtaining a tester nucleic acid molecule for testing for the desired enzymatic activity; c) assaying whether at least a part of the tester nucleic acid molecule facilitates insertion of a sequence into the target sequence; and d) preparing the DNA molecule having the enzymatic domain by producing the molecule comprising the enzymatic activity sequence identified in the tester. In a specific embodiment, there is at least one nucleic acid molecule comprising an enzymatic DNA that provides both phosphodiesterase hydrolysis and phosphodiesterification functions identified by a method described herein.

Additional objects, advantages and novel features of the invention will be set forth in part of the description that follows, and in part will become apparent to those skilled in the art upon examination of the following specification or may be learned by practice of the invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a schematic diagram showing a monofilament AE-DNA as well as a target DNA strand, wherein the “target DNA” is also a single stranded DNA sequence that mimics a double strand DNA by virtue of its ability to fold back upon itself, forming Watson-Crick base pairs by complementary anti-parallel DNA sequences.

FIG. 2 is a schematic diagram illustrating a unilateral reaction comprising cleavage and re-ligation reactions between the monofilament and the target DNA strand shown in FIG. 1.

FIG. 3 is a schematic diagram showing a specific test sequence demonstrating the unilateral reaction.

FIG. 4 is a schematic diagram showing a double stranded AE-DNA of the instant invention as well as a double stranded target DNA.

FIG. 5 is a schematic diagram demonstrating a specific sequence for a double stranded AE-DNA of the instant invention.

FIG. 6 is a schematic diagram showing a hybrid structure resulting from cleavage of the double stranded target DNA shown in FIG. 4 which a double stranded AE-DNA of the present invention.

FIG. 7 illustrates the four DNA sequence components of a bilateral AE-DNA.

FIG. 8 illustrates the four DNA sequence components of a unilateral AE-DNA.

FIG. 9 illustrates another order of DNA functional unit sequences of a bilateral AE-DNA 536 including an EC sequence 502, and HEP sequence 504 and an ALE sequence 503 but lacking a TFO sequence.

FIG. 10A shows another alteration of the DNA sequence components, wherein an AE-DNA 636 has a first strand 621 of nucleotides comprising in order from a 5′ end 673 to a 3′ end 641 an EC sequence 602, first HEP sequence 604 and a TFO sequence 601.

FIG. 10B shows a second semi-bilateral AE-DNA 736 having a reverse polarity from that of the first semi-bilateral AE-DNA 636 wherein similar numbers correspond to similar parts (i.e. second HEP sequence 614 of the first semi-bilateral AE-DNA 636 corresponds to a second HEP sequence 714 of the second semi-bilateral AE-DNA 736).

FIG. 11 illustrates one specific exemplary embodiment of a “padlock exchange” reaction, as described herein.

FIG. 12 illustrates a second specific exemplary embodiment of a “padlock exchange” reaction, as described herein.

Other objects, features and advantages of the present invention will become apparent from the following detailed description. It should be understood, however, that the detailed description and the specific examples, while indicating the preferred embodiments of the invention, are given by way of illustration only, since various changes and modifications within the spirit and scope of the invention will become apparent to those skilled in the art from this detailed description.

### DETAILED DESCRIPTION OF THE PREFERRED EMBODIMENTS

In keeping with long-standing patent law convention, the words “a” and “an” when used in the present specification in concert with the word comprising, including the claims, denote “one or more.”

The practice of the present invention will employ, unless otherwise indicated, conventional techniques of molecular biology, microbiology, recombinant DNA, and so forth which are within the skill of the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), ANIMAL CELL CULTURE (R. I. Freshney, Ed., 1987), the series METHODS IN ENZYMOLOGY (Academic Press, Inc.); GENE TRANSFER VECTORS FOR MAMMALIAN CELLS (J. M. Miller and M. P. Calos eds. 1987), HANDBOOK OF EXPERIMENTAL IMMUNOLOGY, (D. M. Weir and C. C. Blackwell, Eds.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987), CURRENT PROTOCOLS IN IMMUNOLOGY (J. E. coligan, A. M. Kruisbeek, D. H. Margulies, E. M. Shevach and W. Strober, eds., 1991); ANNUAL REVIEW OF IMMUNOLOGY; as well as monographs in journals such as ADVANCES IN IMMUNOLOGY. All patents, patent applications, and publications mentioned herein, both supra and infra, are hereby incorporated herein by reference.

A skilled artisan recognizes that there is a conventional single letter code in the art to represent a selection of nucleotides for a particular nucleotide site, and any of these may be used herein. For example, R refers to A or G; Y refers to C or T; M refers to A or C; K refers to G or T; S refers to C or G; W refers to A or T; H refers to A or C or T; B refers to C or G or T; V refers to A or C or G; D refers to A or G or T; and N refers to A or C or G or T. In other embodiments nucleotide analogs are used, such as compounds similar to one of the four DNA bases (adenine, cytosine, guanine, and thymine) but having a different composition and, as a result, different pairing properties. For example, 5-bromouracil is an analog of thymine but sometimes pairs with guanine, and 2-aminopurine is an analog of adanine but sometimes pairs with cytosine.

The term “activated” as used herein refers to a compound, such as one comprising a phosphate, that comprises a high energy group conferring the ability to unite spontaneously with another molecule. In particular embodiments, the activated molecule is free to ligate to an available 3′ hydroxyl group in an efficient manner. In specific embodiments, a leaving group separates from a compound, which renders a reaction energetically favorable. For example, in nature a diphosphate is removed from a triphosphate, which provides energy for ligation. In specific embodiments utilized herein, however, the term refers to an activated phosphor-imidazolide compound, wherein the leaving group is the imidazolide. In some embodiments, other activated leaving groups are utilized, such as 1-methylimidazole (Kanavardi et al., 1989) or a pyrophosphate group (Rohagti et al., 1996), i.e. a DNA 5′-triphosphate. In alternative embodiments, in lieu of an activated ligatable end, the energy for a reaction is provided by ATP, GTP, TTP, or CTP. For example, the AE-DNA end may comprise a monophosphate end without a leaving group, and the energy is provided by ATP, such as would be provided in a buffer solution.

The term “autocatalysis” as used herein refers to a nucleic acid molecule being able to undertake its own nucleic acid mechanism, such as transesterification, cleavage, and/or ligation. A nucleic acid molecule comprising autocatalysis activity is termed autocatalytic. In a specific embodiment, the autocatalysis mechanism utilizes phosphodiesterase hydrolysis and phosphodiesterification functions.

The term “disease of genetic origin” as used herein refers to a disease or medical condition that is directly or indirectly the result of a particular characteristic of a nucleic acid, such as a mutation in a gene that encodes a gene product. In specific embodiments, a mutation or multiple mutations indirectly or directly renders a gene product in a cell ineffective (because of altered activity, level, or both, for example) or produced at undesirable levels (either too little or too much) to provide a needed function in a cell of the organism in which it is comprised. A skilled artisan recognizes that a mutation can indirectly affect a level or activity of a particular gene product, such as a mutation in another gene product that regulates expression or activity of the particular gene product.

The term “genome” as used herein is defined as the collective gene set carried by an individual, cell, or organelle.

The term “Hoogsteen” base pairing as used herein refers to two nucleotides forming two hydrogen bonds with one another in a manner involving the N7 atom of the purine ring rather than the N1 atom (as occurs in Watson-Crick base pairing). Thus, it is an alternative geometry of bonding between two nucleotides, such as between an adenine and a thymine. A skilled artisan recognizes that the Hoogsteen geometry is favorable for A-T base-pairs in solution. Hoogsteen G-C base pairs have only two hydrogen bonds, and, therefore, protonation is essential for pairing. Furthermore, Hoogsteen base pairing occurs in the major groove of duplex DNA. The binding of a homopurine triplex binds in an antiparallel manner, whereas homopyrimidine triplex binds in a parallel manner.

The term “phosphodiesterase hydrolysis activity” as used herein is defined as a hydrolytic cleaving of a phosphodiesterase bond. This hydrolytic cleavage results in a 3′ hydroxyl group and a 5′ phosphate group.

The term “phosphodiesterification activity” as used herein is defined as condensation between a free 3′-hydroxyl group and a free phosphate group resulting in the reformation of a phosphodiesterase bond.

The term “physiological conditions” as used herein refers to conditions found in a cellular environment or that mimic those found in a cellular environment. The conditions relate to particular pH, concentration of solutes, temperature, and so forth. In a specific embodiment, this includes potassium chloride at about 130-140 mM, pH at approximately 7.4, and the presence of divalent cations.

The term “sequence homologous to a human genomic sequence” refers to nucleotide sequence comprising homology to a sequence located in a human genome, wherein the sequence is at least about 70% homologous, about 75% homologous, about 80% homologous, about 85% homologous, about 90% homologous, about 93% homologous, about 95% homologous, about 97% homologous, or about 99% homologous to the genomic sequence.

The term “therapeutic alteration” as used herein refers to a nucleotide sequence that comprises a particular nucleotide or multiple nucleotides that, when exchanged with the homologous sequence comprising a defective nucleotide sequence in, for example, a genome of a cell of an individual, the particular nucleotide or multiple nucleotides changes the defect and, preferably, improves, ameliorates, prevents, reduces, or cures at least one symptom of any disease or medical condition in the individual that indirectly or directly results from the defect.

The term “transesterification” as used herein is defined as one phosphate ester converted directly to another with an leaving group intermediary, such as, for example, a imidazole intermediary. Given that bonds are exchanged directly, energy is thus conserved, and the reaction requires no input of energy, such as from hydrolysis of ATP or GTP.

The present invention provides a sequence-specific unilateral and bilateral nucleic acid exchange reactions that permit the exchange of nucleic acid strands. In specific embodiments, the nucleic acid strand exchange reactions are useful for genetic correction of a nucleotide sequence, such as a gene in vivo, for a therapeutic purpose. The genetic correction comprises exchanging at least one nucleotide with the analogous defective nucleotide in the sequence in vivo in an individual, such as a mammal.

A skilled artisan recognizes that the present invention is useful for exchanging one nucleotide sequence for another, wherein in some embodiments the nucleotide sequence to be replaced comprises an undesirable character. The undesirable character could be one or more nucleotides. The undesirable character could comprise a modification of a nucleotide or multiple nucleotides compared to a wild-type nondefective sequence. In specific embodiments, the undesirable character is an alteration in a nucleotide sequence. In further specific embodiments, the alteration in the nucleotide sequence is a mutation, such as a point mutation, a frameshift, a deletion, an inversion, a combination thereof, and so forth. In other embodiments, the undesirable character to be replaced comprises a wild type nucleotide sequence that encodes an undesirable gene product, wherein-it would be preferable to reduce, inhibit, prevent, impede, or eliminate the activity or level of the undesirable gene product. This may occur by affecting regulatory sequences (transcriptional, post-transcriptional, translational, or post-translational) for the expression of the undesirable sequence, or it may affect nucleotide sequence encoding amino acid residue(s) important for function, such as in an active site, a functional domain, or a region that affects structure of the gene product.

As in the case for the unilateral action, the combination of these four units (or a subset thereof) for the bilateral reaction has been termed the “Active Exchangeable DNA” (AE-DNA). The term “unilateral AE-DNA” will be used to signify an AE-DNA that is functional as a monofilament (such as wherein the DNA strand is folded back upon itself to mimic a double stranded DNA molecule). The term “bilateral AE-DNA” will be used to signify an AE-DNA comprised of two monofilament DNA strands bound to one another through a region of complementary base pairs of the HEP 303.

A skilled artisan recognizes that the monofilament strand(s) of the present invention is a nucleic acid that may be comprised of deoxyribonucleotides, ribonucleotides, or a mixture thereof. There are significant differences between the DNA/RNA embodiments of compositions described herein and others known in the art, particularly those described in U.S. Pat. Nos. 5,565,350; 5,731,181; 5,760,012; 5,756,325; 5,795,972; 5,871,984; and 5,888,983. Besides the noticeable structural differences, the mechanism employed to effect site-specific genetic change is substantial as well. Whereas the DNA/RNA comprised AE-DNA constructs described herein comprise multiple components that serve different functions (i.e. catalytic, TFO, activated ligatable group, region of homology), the chimeric DNA/RNA molecules described in those patents are simple structures designed to bind to themselves by complementary sequences that form Watson-Crick base pairs. Their chimeric DNA/RNA molecules then bind to the gene target sequence, again by simple Watson-Crick base pairing rules. It is postulated that the eukaryotic cellular equivalence of a protein that serves similar functions to the Rec A protein in E. coli is involved in the binding of the chimeric molecules to target duplex DNA. The Rec A protein binds to single stranded DNA and facilitates this single stranded DNA to hybridize with a homologous duplex DNA regions. It is postulated that this Rec A-mediated process, as well as other cellular processes involved with homologous recombination, are essential for the function of the chimeric DNA/RNA mediated site specific repair described therein.

In contrast, AE-DNA mechanisms participate in target localization by both triplex forming oligonucleotide strategies as well as D-loop strand invasion mechanisms, and the single stranded nucleic acid of AE-DNA autocatalytically inserts itself into the duplex DNA target after this covalently linked structure (through transesterification) is then recognized by the cellular machinery as a duplex DNA break and initiates cellular DNA repair machinery. This coupled with mismatch/excision repair completes the site directed mutogenesis process. Although the final common pathway that the cell uses to repair mismatched bases may be similar in both methods, the intermediary structures between these two methods are vastly different. That is, the AE-DNA employs a covalently linked intermediary with a single strand DNA break, whereas the chimeric DNA/RNA hybrid relies solely on traditional Watson-Crick base pairing with the DNA duplex targeted interest.

A skilled artisan recognizes that the methods and compositions described herein are utilized in vitro and/or in vivo under physiological conditions. Although a skilled artisan would recognize the specific requirements of physiological conditions, in exemplary embodiments they are an aqueous solution of about 140 mM potassium chloride, about 15 mM Hepes buffer at pH 7.4, about 1-20 mM MgCl₂, ATP to about 0.2 mM, Zinc from 1 mM to about 2 mM, histidine at about 1 mM to about 2 mM, and about 1 mM to about 5 mM spermine, such as per 50 microliters of solution, preferably carried out at about 37° C. Other divalent cations listed herein are preferably between about 1 mM to about 2 mM.

In some embodiments, a monovalent cation, a divalent cation, a mixture of monovalent cations, a mixture of divalent cations, or a a mixture of divalent and monovalent cations is utilized in the methods and compositions. Exemplary monovalent cations include Na⁺, K⁺, Li⁺, or H⁺. Exemplary divalent cations include members of the Irving-Williams series (Ba²⁺, Sr²⁺, Ca²⁺, Mg²⁺, Mn²⁺, Fe²⁺, Co²⁺, Ni²⁺, Cu²⁺, Zn²⁺) as well as Pb²⁺ and Cd²⁺. Exemplary concentrations of the monovalent and/or divalent cations include between about 1 mM and 150 mM. In specific embodiments of the present invention, the divalent cation acts as a bridge between the phosphate group and a phosphate group from the invading single strand nucleic acid.

The invention is usable to genetically alter biological elements with their accompanying disease processes, examples of which are listed below.

| GENETIC ALTERATION | DISEASE PROCESS |
|---|---|
| LIPID METABOLISM | |
| Lipoprotein Lipase | Hypertriglycerdemia |
| Apolipoprotein E | Hyperlipoproteinemia |
| LDL receptor | Hypercholesterolemia, altered |
| | Lipoproteins |
| HMG Co-A reductase | Hyperlipoproteinemia |
| Apolipoprotein B mRNA editing | Hypercholesterolemia |
| protein | |
| Apolipoprotein A1 | Hyperlipoproteinemia |
| Vascular Endothelial Growth Factor | Atherosclerosis/Cancer invasion |
| (induces angiogenesis by ischemia) | |
| Lecithin Cholesterol acyltransferase | Atherosclerosis, Increase HDL |
| (LCAT) | Chol |
| CARDIAC | |
| Alpha-tropomyosin | Hypertrophic Myocardiopathy |
| Cardiac Troponin T | Hypertrophic Myocardiopathy |
| Beta-Myosin Heavy Chain | Hypertrophic Myocardiopathy |
| ANTICOAGULATION | |
| Plasminogen activators | Local Anticoagulation |
| Hirudin | Local Anticoagulation |
| Urokinase | Local Anticoagulation |
| VASCULAR/HYPERTENSION | |
| Nitric Oxide Synthase | Neointimal vascular lesion |
| | Arterial Nitric oxide abnormalities |
| Nitric Oxide Synthetase | Vascular Disease, Impotence, |
| | Neuron injury |
| Prostaglandin H Synthase | Local Arterial Injury |
| Cyclin/cyclin dependent kinase | Vascular smooth muscle cell |
| inhibitor P21 | proliferation |
| | After angioplasty |
| Fibroblast Growth Factor | Hypertension |
| Kallikrein | Hypertension |
| HEMATOLOGY | |
| Clotting Factor Deficiencies | |
| Clotting Factor Factor IX | Hemophilia B |
| Deficiencies Factor VIII | Hemophilia A |
| Protein C | Protein C Deficiency |
| Erythropoitin | Beta-Thalessemia |
| Gamma-Globin | Sickle-Cell Anemia |
| Delta-globin | Sickle-Cell Anemia |
| HST-11 Fibroblast Growth Factor-4 | Thrombocytopenia |
| Thrombopietin | Thrombocytopenia |
| Interleukin-11 | Thrombrocytopenia |
| ONCOLOGY | |
| Beta Transforming Growth Factor | Cancer |
| P-53 | Breast, Brain, Colon, Kidney, |
| | Ovary |
| P-21 | Osteosarcoma |
| C-CAM-1 Tumor Suppressor | Prostrate, solid Tumors |
| Multi-Drug Resistance Protein | Most tumors |
| DNA methyltransferase inhibitors | Most tumors |
| Macrophage Colony Stimulating | Melanoma |
| Factor | |
| Manganese Superoxide Dismutase | Squamous cell carcinoma |
| Tumor Necrosis Factor-alpha | Most tumors, wasting |
| Bcl-x | Leukemia, Lymphoma |
| Epidermal Growth Factor | Lung, Brain, Breast |
| Prostate Specific Antigen Promoter | Prostate |
| Von Hippel-Lindau tumor | Renal Carcinoma |
| suppressor | |
| Anti erbB2 single-chain | Breast, Ovarian Carcinoma |
| immunoglobulin | |
| c-myb oncogene antisense | CNS tumors |
| Cytokines | Tumors |
| DIABETES | |
| Glucokinase | Diabetes, Hyperglycemia |
| Preproinsulin II | Diabetes |
| Vasoactive Intestical Protein | Diabetes |
| Calcitonin gene related peptide | Diabetes |
| NEURAL DISEASE | |
| Tyrosine Hydroxylase | Parkinson's Disease |
| GTP cyclohydrolase I | Parkinson's Disease |
| Nerve Growth Factor | Spinal injury, Parkinson's |
| Fibroblast Growth Factor-4 | Amyothophic Lateral Sclerosis |
| Dystrophin | Duchenne's Muscular Dystrophy |
| Beta-endorphin | Chronic pain |
| Ciliary Neurotrophic Factor | Huntington's Disease, ALS |
| TRAUMA | |
| Alpha Fibroblast Growth Factor | Wound Healing |
| Platelet-Derived Growth Factor | Wound Healing |
| METABOLIC | |
| Glucocerebrosidase | Gaucher's Disease |
| Phenylalanine hydroxylase | Phenylketonuria (PKU) |
| RARE METABOLIC | |
| Fumarylacetonacetate hydrolase | Tyrosinaemia |
| (FAH) | |
| Copper transporting protein | Wilson's Disease |
| Aspartyglucosaminidase | Aspartylglucosaminuria |
| Beta-glucuronidase | Sly's syndrome |
| Adeosine deaminase | Severe Combined |
| | Immunodeficiency (SCID) |
| Purine nucleoside phosphorylase | PNP deficiency (SCID |
| (PNP) | |
| Alpha-Iduronidase | Hurler's Disease |
| Iduronate-2-sulfatase | Hunter Syndrome |
| Alpha-galactosidase-A | Fabry disease |
| P-glycoprotein | Fabray disease |
| Ornithine-delta-aminotransferase | OAT deficiency |
| (OAT) | |
| Acid Maltase | Pompe's Disease |
| Branched-chain keto acid | Maple Syrup Urine Disease |
| dehydrogenase | |
| Glycogen Phosphorylase | Glycogen Storage |
| Arginase isozyme | Liver arginase deficiency |
| Alpha-1 fucosidase | Fucosidosis |
| OPHTHALMOLOGY | |
| Tyrosinase | Retinal photoreceptor, rod |
| | Deficiencies |
| Ciliary Neurotrophic Factor | Retinal photoreceptor degeneration |
| Neural Growth Factors | Retinal photoreceptor degeneration |
| Beta phosphodiesterase | Retinal photoreceptor degeneration |
| OBESITY/G.I. | |
| Leptin | Obesity |
| Transforming growth factor-beta 1 | Colitis/Inflammatory bowel |
| ICAM's | Colitis/Inflammatory bowel |
| RESPIRATORY | |
| Cystic Fibrosis Transmembrane | Cystic Fibrosis |
| Regulator | |
| Alpha1-anti-trypsin | Emphysema |
| Prostaglandin synthase | Asthma |
| RHEUMATOLOGIC | |
| Ostepetegrin | Osteoporosis, Bone Tumors |
| Interleukin-1 Receptor Antagonist | Osteoarthritis |
| Interleukin-1 Receptor Antagonist | Rheumatoid Arthritis |
| Decorin | Glomerulonephritis |
| ANTI-INFLAMMATORY | |
| Interleukin-4 | Anti-inflammatory |
| Interleukin-10 | Anti-inflammatory |
| Interleukin-14 | Anti-inflammatory |
| HEARING LOSS | |
| Cholecystokinin | Audiogenic seizures |
| NADPH-oxidase | Chronic Granulomatous Disease |
| Transglutaminase1 | Lamellar ichthyosis |

A skilled artisan recognizes how to identify and obtain sequence to be exchanged with the target defective sequence, such as from sequence databases. One publicly available database is the National Center for Biotechnology Information's GenBank database, which is well known in the art. A skilled artisan identifies the therapy to be applied, pursuant to the medical condition of the individual, and locates the appropriate nucleotide sequence in the database. The sequence is preferably from the same organism as is being treated, although interspecies sequences may also be effective, and a skilled artisan knows standard means in the art for testing this. Following obtaining the sequence, such as by polymerase chain reaction methods well known in the art and optionally sequencing to ensure its integrity, the skilled artisan utilizes the sequences in methods and compositions described herein. Although a skilled artisan would know which sequences to obtain for a therapy, as well as their identity from a database, exemplary sequences include p53 (M14695; SEQ ID NO:11) for cancer treatment and gamma-globin (X55656; SEQ ID NO:12) for sickle cell anemia treatment. In specific embodiments, the sequence is obtained and then mutagenized so as to comprise a therapeutic mutation (such as to render an oncogene ineffective).

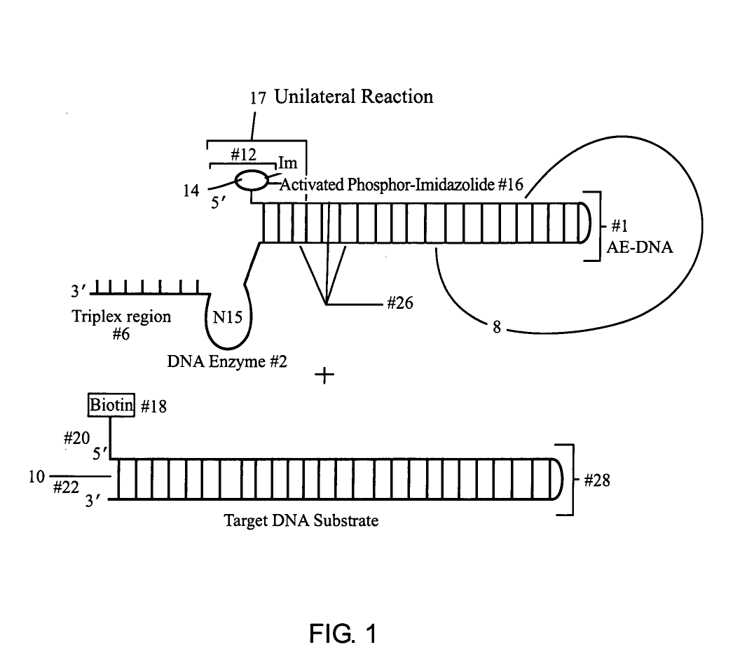

As shown in FIGS. 1-3, the unilateral reaction described herein demonstrates the feasibility of (1) monofilament nucleic acid exchange without using catalytic protein assistance; and (2) the ability to obtain sequence information regarding the actual nucleic acid enzymatic component that promotes this type of exchange reaction. The catalytic nucleic acid component of this invention in specific embodiments possesses both phosphodiesterase-like activity (DNA cleavage ability) and DNA ligase-like activity to be effective. In other embodiments, the catalytic nucleic acid component of this invention possesses transesterification activity. There has never been a demonstration in the literature of a DNA sequence that is capable of performing both of these catalytic reactions. The unilateral reaction is the first to identify DNA sequences of interest that can fulfill this role.

In one embodiment of the present invention, a system for identifying catalytic DNA strand exchange reactions comprises two separate oligonucleotides. The first oligonucleotide is preferably immobilizable (such as, for example, being biotin-linked) and is designed to fold back upon itself in a complementary inverted structure that favors Watson-Crick duplex base pairing, preferably mimicking a duplex DNA structure. The other oligonucleotide termed the “activated enzymatic DNA” (AE-DNA) represents an oligo that contains, preferably, all four of the previously described components. This includes the enzymatic 15 base pair (an exemplary size) segment, the triplex binding region, the activated imidazolide leaving group, and the area of homology with the target duplex DNA of interest.

These reactions are screened by first binding the biotin-containing oligonucleotide to a streptavidin column. The AE-DNA oligonucleotide is then added with appropriate buffers and incubated at various time points, followed by stringent column washing. Strand exchange reactions are then recovered after displacing the biotin oligonucleotide from the column, and PCR amplifications are carried out with primers from the 5′ end of the biotin oligonucleotide and the 3′ end of the AE-DNA oligo. Only DNA strands that have been exchanged will comprise both components from each oligo (5′ from the biotin oligo and a 3′ from the AE-DNA oligo) and can be amplified by PCR.

I. The Unilateral Reaction

FIG. 1 illustrates an exemplary basic functional unit that comprises a DNA sequence that carries out the exchange reaction steps. The basic functional unit described is termed unilateral “Activated Exchangeable DNA” 1, hereinafter referred to as AE-DNA 1. AE-DNA 1 comprises, preferably and for example, four sequence elements of particular interest, each of which defines a specific function that permits for the exchange reaction to occur. At least some of the actual sequences that comprise the various functions will vary depending upon the target DNA substrate that is used in the exchange reaction. That is, a sequence to be exchanged with the target DNA substrate will be selected based on the desired target DNA to be exchanged. For example, if a defective p53 sequence is to be remedied, the AE-DNA will comprise a homologous p53 sequence that does not comprise the defect.

The target DNA substrate, hereinafter sometimes referred to as target DNA 28 or substrate 28, is a nucleotide sequence that the AE-DNA 1 is designed to “recognize” (bind with) and exchange DNA sequences. These DNA exchange reactions involve both phosphodiesterase hydrolysis reactions (cleavage) and subsequent phosphodiesterification reactions (ligation) between two DNA monofilaments. The two DNA monofilaments (AE-DNA 1 and the target DNA 28) are purposefully designed to be compatible with each other so as to effectively promote these exchange reactions. In other words, if a desired DNA target is identified, relevant sequences of the AE-DNA can be altered accordingly to promote an exchange reaction with that particular target DNA of interest. It is also important to note that these reactions were designed such that the “target DNA” will fold back upon itself forming Waston-Crick base pairing to mimic a double stranded DNA target.

The AE-DNA 1 will preferably include four components (although fewer than four may be utilized), which comprise a first sequence designed to confer triplex forming oligonucleotide (“TFO”) functions to the target DNA 28, this first sequence hereinafter being referred to as TFO sequence 6. Triplex forming oligonucleotide functions are well-known in the literature and rely on Hoogsteen base pairing to a double stranded DNA target. A skilled artisan recognizes that TFOs bind in the major groove of duplex DNA with high specificity and affinity (Vasquez and Glazer, 2002; Giovannangeli and Helene, 2000). Although in many embodiments the triplex forming structure requires homopurines, alternative means are known, such as those utilizing 2-aminoquinazoline C-nucleotide (Li et al., 2003). Other embodiments utilize triplex forming oligonucleotides that are homopyrimidines. A skilled artisan recognizes that for binding of the TFO in an antiparallel fashion, a homopurine TFO would preferably be used, and for binding of the TFO in a parallel fashion, a homopyrimidine TFO would preferably be used. In some embodiments conjugates for the TFOs are utilized, such as the exemplary conjugate of a dipyrido[3,2-a:2′,3′-c]phenazine-ruthenium (II) complex and a triple helix-forming oligonucleotide (Grimm et al., 2001). A size range for a TFO would be from about 10 nucleotides to about 30 nucleotides.

The second sequence is the “DNA enzyme” component (“enzymatic component or EC”), this second sequence hereinafter being referred to as the EC sequence 2. Preferably, the EC sequence 2 has about 15 nucleotides, although a range of about 5 nucleotides to about 30 nucleotides is useful. The TFO sequence 6 and the EC sequence 2 in the unilateral AE-DNA 1 construct are in cis-orientation to each other with the TFO sequence 6 occupying the most distal 3′ end of the conjoined sequences described thus far. In the methods described herein, the a sequence is tested for having the enzymatic activity. The sequence to be tested may be obtained in a variety of ways, such as obtaining a known sequence or alternatively by producing random sequences and testing them. Random sequence oligonucleotides are generated by well-known means in the art. The various sequences to be tested are inserted into a AE-DNA system for testing, as described herein, and tested for phosphodiesterase hydrolysis and phosphodiesterification capability.

The EC sequence 2 is itself immediately 3′ in orientation to the third sequence, namely the “Homologous Exchangeable Piece” (“HEP”), hereinafter referred to as the HEP sequence 8. The HEP sequence 8 is essentially a homolog of the target DNA 28, except that the HEP sequence preferably comprises at least one nucleotide base sequence differing at one or more nucleotide base(s) from the nucleotide base sequence of the target DNA substrate. The HEP sequence may also contain some base sequences that can participate in triplex binding as well. After an exchange reaction between the AE-DNA 1 and the target DNA 28 has occurred, these nucleotide base pair changes preferably alter the target DNA, in order to achieve a desired therapeutic effect. In preferred embodiments, at least a portion of the HEP 8 that comprises the at least one nucleotide difference is exchanged with at least a portion of the target DNA. During the unilateral reaction, the HEP sequence 8 is folded back upon itself, mimicking a double stranded DNA. While maintaining this folded configuration, the homology that exists between the target DNA 28 and the HEP sequence 8 allows for inter-strand base pairing to occur.

Located at the 5′ end of the homolog is, preferably, for example, an activated phosphate group 12 comprising a phosphate group 14 covalently linked to an imidazolide group 16 to form an activated phosphor-imidazolide group 16. The activated phosphor-imidazolide group has previously been described (Cuenoud et al., 1995) and is capable of ligating itself to a free 3′ hydroxyl end of a DNA molecule. This terminal 5′ activated phosphor-imidazolide group 16 represents the fourth sequence that is termed the “Active Ligatable End” (“ALE”), this fourth sequence hereinafter being referred to as ALE sequence 17. At a minimum, sequence 17 comprises the imidazolide group, the 5′ phosphate and the most distal 5′ nucleotide base.

In some embodiments, other activated leaving groups are utilized, such as 1-methylimidazole (Kanavardi et al., 1989) or a pyrophosphate group (Rohagti et al., 1996), i.e. a DNA 5′-triphosphate. In alternative embodiments, in lieu of an activated ligatable end, the energy for a reaction is provided by ATP, GTP, TTP, or CTP. For example, the AE-DNA end may comprise a monophosphate end without a leaving group, and the energy is provided by ATP.

In specific embodiments, it is useful to have the HEP adjacent to the EC, so that the EC is conveniently accessed upon homology identification between the two sequences. It is also preferable to have the ALE adjacent to the position desired to cleave and religate or the position for initiation of a transesterification reaction.

As shown particularly in FIG. 1, the target DNA 28 also comprises a folded monofilament DNA that is homologous with the HEP and that also mimics a double stranded DNA. The substrate has both a 3′ end 22 and a 5′ end 20 whereat an “anchor” molecule such as, for example, biotin group 18, is covalently linked to a streptavidin labeled column (not shown). The target DNA functions as a substrate for the unilateral reaction and comprises a stem structure folded in an anti-parallel structure mimicking double stranded DNA.

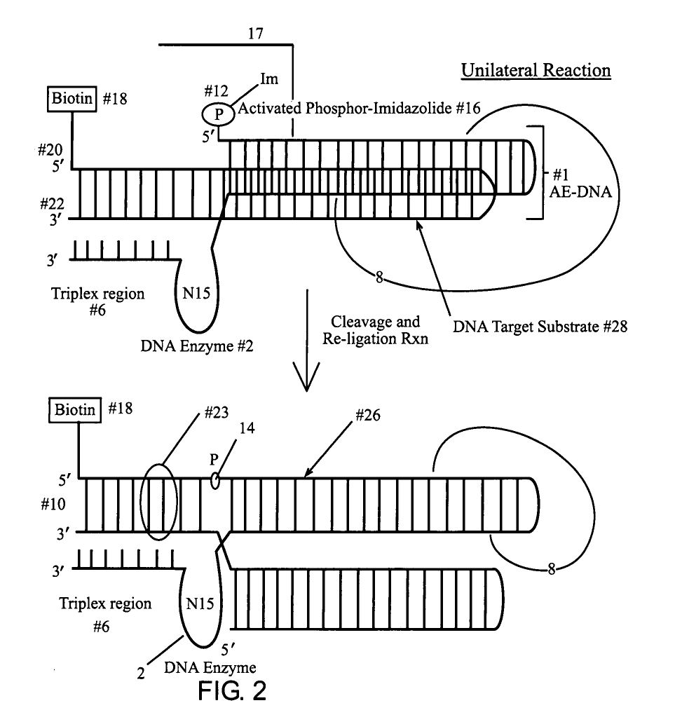

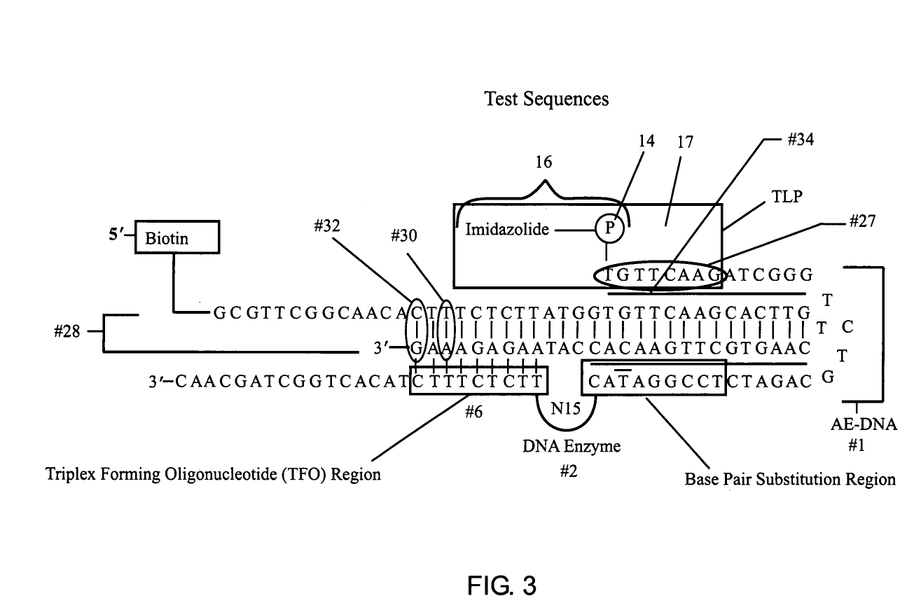

Referring now to FIGS. 2 and 3, the target DNA substrate 28 is supported by the anchor biotin 18 bound to a streptavidin column. When the AE-DNA 1 is added to the column, the triplex forming oligonucleotide component, TFO sequence 6, recognizes and binds to the target DNA 28 by hydrogen bonding according to Hoogsteen rules. With the target DNA substrate attached to a solid matrix via the biotin moiety, the AE-DNA strand 1 can be denatured easily with increasingly stringent conditions using conventional techniques. This unilateral reaction system is designed in such a manner that when the AE-DNA 1 is successfully exchanged by both a cleavage reaction and a subsequent ligation reaction such that it is covalently linked to the target DNA 28, it will remain anchored to the streptavidin column via the target DNA strand and will not be washed away under denaturing conditions (0.1 M NaOH). If the unilateral reaction exchange is not successful, the AE-DNA 1 will not be anchored and is washed away under denaturing conditions. These operating conditions guide the selection of the nucleotide sequences that comprise the EC sequence 2 of the AE-DNA strand 1 and that are capable of catalyzing both the cleavage and re-ligation reactions. Because these reactions are also conducted under physiological conditions, in specific embodiments these functions also are conductable in vivo.

FIG. 3 depicts specific yet exemplary DNA sequences suitable for the described unilateral reaction, as illustrated with SEQ ID NO:1 and SEQ ID NO:2. Specifically, TFO sequence 6 of the AE-DNA strand 1 allows the EC sequence 2 to bind with the correct orientation for the cleavage of the molecule in a sequence-specific manner. Following this reaction step, the HEP sequence 8 is able to exchange DNA strands after the ALE sequence 17 containing the activated phosphate group 16 religates to the cleaved substrate target DNA 28. Both the cleavage and re-ligation reactions are catalyzed by the EC sequence 2, thereby completing the unilateral exchange reaction.

The sequence-specific cleavage is dependent on the terminal 5′ of the ALE sequence that is partially homologous to the 3′ antiparallel strand of the target DNA substrate 28. In a dynamic reaction, the 5′ terminal sequence of the ALE binds to the 3′ sequence of the target DNA substrate 28 and intermittently displaces the original 5′ strand of the target DNA.

The cleavage and the re-ligation of the molecule is catalytically promoted by the 15 nucleotide sequence of the enzyme portion EC sequence 2. Re-ligation of the juncture between the DNA target 28 and the 5′ end of the novel unilateral AE-DNA strand 1 occurs when the activated phosphate group 16 comes into proximity with a 3′ hydroxyl group of the cleaved substrate, resulting in a new hybrid DNA molecule made from the exchange reaction. The new hybrid DNA molecule consists of the most proximal 5′ component of the original target DNA substrate 10 and the entire unilateral AE-DNA molecule 1 except for the loss of the activating imidazolide component 26 that is hydrolyzed during the reaction. The imidazolide component is part of the phosphor-imidazolide group 16. Imidazole is a five membered ring structure that has been covalently linked to the terminal 5′ end of the DNA oligonucleotide molecule (i.e., the AE-DNA molecule).

Operationally, this reaction occurs by first hydrolysis of the phosphodiester backbone of the “target” DNA, thereby leaving a free 3′ hydroxyl group. This free 3′ hydroxyl group is then available to participate in a nucleophile attack to the phosphor-imidazolide group, complementing the re-esterification reaction.

FIG. 3 shows two monofilament DNA strands represented as actual base pair sequences. One exemplary base pair 30 of the sequence is “T-A” wherein “T” symbolizes thymine and “A” symbolizes adenine. The target DNA substrate 28 and the EC sequence 2 are shown. The binding of the TFO sequence 6 to the target DNA 28 according to Hoogsteen binding rules is depicted as an oval around the “G-C” pair 32 wherein “G” symbolizes guanine and “C” symbolizes cytosine. The proximal 5′ end 27 of the unilateral AE-DNA 1 is shown to competitively bind to the 3′ antiparallel sequence of the substrate 28 represented by pairing at 34.

The unilateral exchange reaction of the present invention can be used to determine specific nucleotide sequences comprising the DNA enzymatic component (EC sequence 2). Test results confirm that exchange reactions do occur at precisely the correct junction predicted for the unilateral reaction. The hybrid DNA exchanged products, still bound to the streptavidin column following cleavage and ligation steps, were subsequently isolated and sequenced to reveal the composition of the catalytic units. Exemplary sequences that promote these exchange reactions are provided herein (in specific embodiments, those termed EC-N15-02, EC-N15-10, and EC-N15-78).

A skilled artisan recognizes that, in some embodiments, the unilateral reaction is preferred for in vivo purposes instead of the bilateral reaction, given that in some embodiments a bilateral reaction comprises cleavage of a double strand, which may undesirably elicit DNA repair mechanisms.

II. The Bilateral Reaction

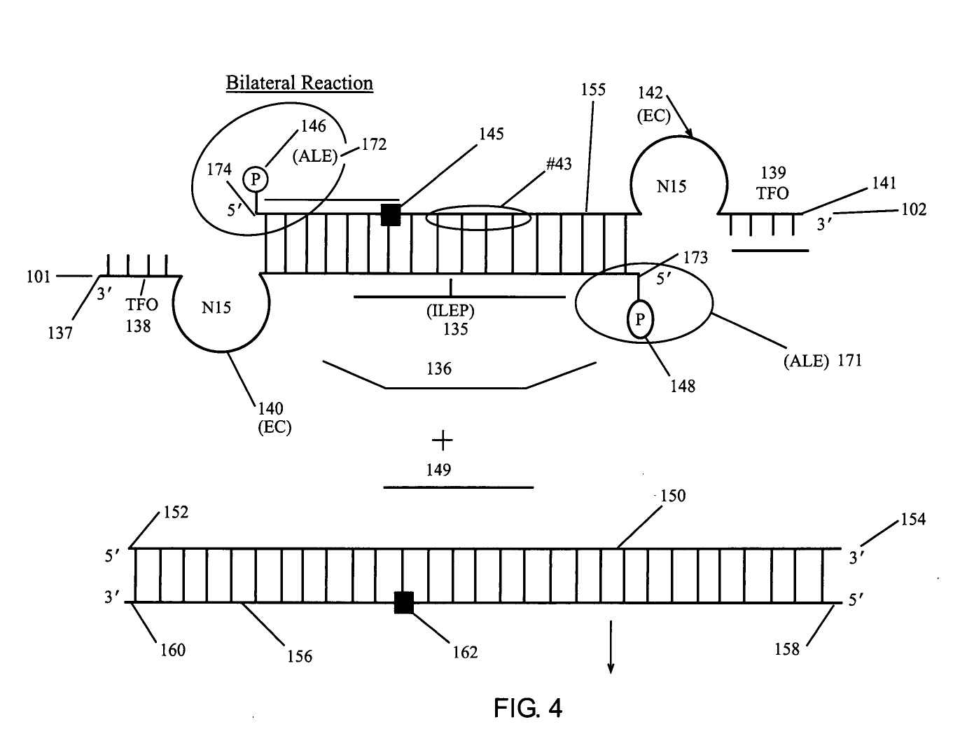

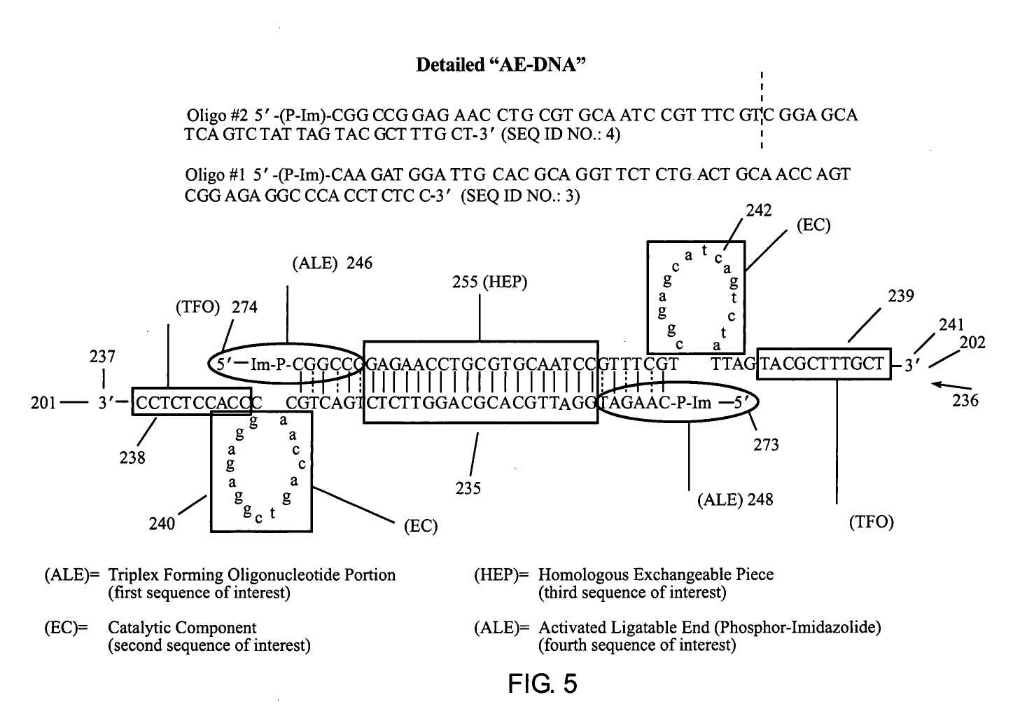

The bilateral reaction is analogous to the unilateral reaction and comprises two hybridized monofilaments of AE-DNA. These duplicate AE-DNA act in a mirror image-like fashion to perform bilateral DNA strand exchange reactions. Referring to FIGS. 4 and 5, the components of a bilateral enzyme 136 are shown that is collectively termed the “bilateral AE-DNA” 136. The bilateral AE-DNA is designed to interact with a target DNA substrate 149 having a double strand configuration. The bilateral AE-DNA consists of two monofilament DNA strands first and second AE-DNA strands, hereinafter referred to as first AE-DNA 101 and second AE-DNA 102, respectively, each of which comprises four nucleotide sequences as well as an activated 5′ phosphate group. This arrangement of the four sequences is similar in arrangement to the unilateral AE-DNA 1. The first AE-DNA 101 starting from the 3′ end 137 comprises a first sequence that is designed to confer triplex forming oligonucleotide binding functions, hereinafter referred to as first TFO 138. The complementary second AE-DNA 102 also comprises a first sequence having triplex forming oligonucleotide binding functions, said sequence hereinafter referred to as second TFO 139 and being situated at the 3′ end 141 of the second AE-DNA 102. Both the first and second TFO 138 and 139 are covalently linked as part of their respective AE-DNA strands to a respective second sequence in cis-orientation, termed first and second enzymatic components, hereinafter referred to as first EC 140 and second EC 142, respectively. The third sequences in each AE-DNA strand, bound to a respective first EC or second EC, are the “first and second homologous exchangeable pieces” hereinafter referred to as first HEP 135 and second HEP 155, respectively. The first and second HEP exhibit a homology with the target DNA 149 and are the components of the bilateral DNA 136 that ultimately become resident in the target DNA upon completing the exchange reactions. Although homologous with the target DNA 149, the first and second HEP 135, 155 differ therefrom by at least one nucleotide base with the target DNA 149. Upon completing the exchange reactions, these differences in nucleotide bases become resident in the target DNA.

Finally, the fourth sequence in each AE-DNA strand 101, 102 are respective “first and second activated ligatable ends” hereinafter referred to as first ALE 171 and second ALE 172, respectively, each occupying the most distal 5′ ends 173, 174 of the respective first and second AE-DNA strands. Each ALE preferably includes at the 5′ terminus a covalently bound activated phosphor-imidazolide group 148, 146 at the 5′ ends of the first and second AE-DNA strands 101, 102; the bilateral AE-DNA 136 is capable of ligation with a free 3′ hydroxyl group of another DNA strand such as a 3′ hydroxyl of the target double stranded DNA 149. As with the unilateral AE-DNA, the nucleotide sequences of the bilateral AE-DNA are selected to be homologous with a target DNA 149.

Referring to FIG. 4, the targeted double stranded DNA 149 that may be an in vivo gene target, for example, the gene for sickle cell anemia or cystic fibrosis, comprises a first strand 150 having a 5′ end 152 and a 3′ end 154. The target DNA further comprises a second strand 156 also having a 5′ end 158 and a 3′ end 160. A target base 162 is the subject nucleotide in the nucleotide sequence of this target DNA requiring replacement. The bilateral AE-DNA 136 includes a corrective nucleotide 145 that will be swapped into the target DNA 149 when reactions between the AE-DNA 149 and the target DNA are completed. Thus, in this exemplary reaction, nucleotides 145 and 162 are nonidentical.

Referring to the detailed exemplary FIG. 5 in view of FIG. 4, the sequence for an exemplary bilateral AE-DNA 236 is shown having mirror-image first and second AE-DNA strands 201 and 202 arranged in a complementary configuration. Exemplary nucleotide sequences for the two strands is provided as follows:

(SEQ ID NO:3) Oligo #1 (first AE-DNA strand 201): 5′-(P-Im)-CAA GAT GGA TTG CAC GCA GGT TCT CTG ACT GCA ACC AGT CGG AGA GGC CCA CCT CTC C-3′

(SEQ ID NO:4) Oligo #2 (second AE-DNA strand 202): 5′-(P-Im)-CGG CCG GAG AAC CTG CGT GCA ATC CGT TTC GTC GGA GCA TCA GTC TAT TAG TAC GCT TTG CT-3′.

Each respective strand 201, 202 includes in order from a 3′ end 237, 241 to a 5′ end 273, 274 the four sequences in order: TFO 238, 239; EC 240, 242; HEP 235, 255 and ALE 248, 246. Looking to the specific exemplary sequences shown, it is seen that the ALE sequences are at least partially homologous with the 3′ end of the HEP of the opposing AE-DNA strand. In other words, the first ALE 248 of the first strand 201 is at least partially homologous with the 3′ end of the second HEP 255 of the second AE-DNA strand 202. Similarly, the second ALE 246 of the first strand 202 is at least partially homologous with the 3′ end of the first HEP 235 of the first AE-DNA strand 201. In FIG. 5, complementary nucleotide bases between the two strands are illustrated as solid lines, and noncomplementary nucleotide bases between the two strands are illustrated as dashed lines.

It is important to note that the nucleotide sequences for the first and second TFO 238, 239; the first and second EC 240,242; the first and second HEP 235, 255 and the first and second ALE 248, 246 are selected for homology depending on the nucleotide sequence of the target DNA (e.g. sickle cell gene, an oncogene, and so forth). Preferably, the sequences for the first TFO 238, first EC 240 and first ALE 248 will differ from those of the second TFO 239, second EC 242 and second ALE 246. The selection of nucleotide sequences for the EC 240,242 is limited to those sequences exhibiting enzymatic properties (catalytically assist with the cleavage and re-ligation reactions of the bilateral and unilateral AE-DNA exchange reactions). Because these sequences are limited, they may be utilized in a variety of bilateral AE-DNA constructs designed for diverse target DNAs.

As most can clearly be seen in FIG. 5, the sequences of the first and second HEP 235, 255 are preferably fully complementary to one another in order to promote annealing between the two strands 201,202, although in some embodiments there may be less than 100% complementarity, such as about 70%, about 75%, about 80%, about 85%, about 90%, about 95%, about 97%, or about 99%. The first HEP 235 and second ALE 246 also have a region wherein at least some of their individual sequences complement one another, as do at least some of the sequences of the second HEP 255 and first ALE 248, thereby further assisting in annealing and in promoting the stability of the bilateral AE-DNA 236.

The bilateral reaction is analogous to the unilateral AE-DNA reaction described hereinabove, except that the bilateral AE-DNA, having two strands that exhibit a region of complementarity, does not require the folded configuration assumed by the unilateral AE-DNA. Moreover, whereas the reaction involving unilateral AE-DNA involves one cleavage and re-ligation reaction, with the bilateral AE-DNA two such cleavage and re-ligation reactions are required. Cleavage and re-ligation of the double strand occurs, in some embodiments, at a 5′ proximal location relative to the target sequence to be exchanged.

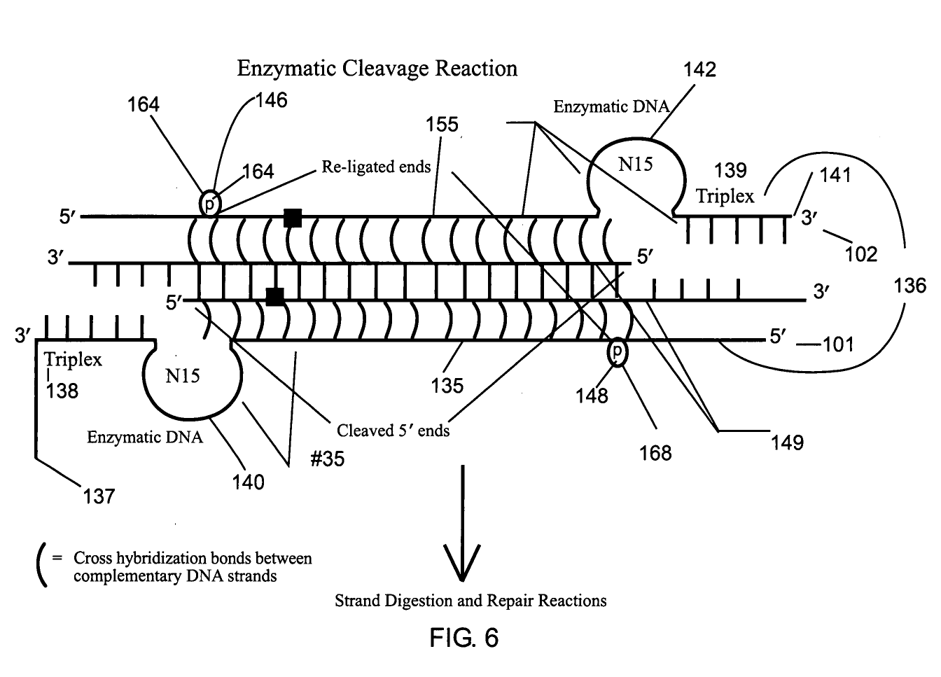

Referring now to FIG. 6, a particular targeted double stranded DNA 149 is shown after cleavage and re-ligation reactions have been mediated by a bilateral AE-DNA 136. Cleavage points 168, 164 occur at the sites where the activated phosphate groups 148, 146, respectively, react with the target DNA 149. Both cleavage and re-ligation reactions occur at these sites. Hydrolysis of the activated phosphate groups causes a loss of the imidazolide groups during the re-ligation between the strands of the target DNA and the strands of the bilateral AE-DNA. The newly formed hybrid structure is a composite of target DNA and the HEP sequences of the AE-DNA resulting from the two symmetrical cleavage and re-ligation reactions.

In FIG. 6, completed junctions at the cleavage points 168, 164 have been shown with the phosphate groups 148, 146 still attached to the AE-DNA 136 for illustrative purposes only. In reality, the phosphodiesterase backbone of the DNA would be re-established and seamless following the re-ligation reactions. By this time, the hybrid double stranded DNA would be continuous along each strand, comprising in part the original target DNA and in part the 5′ to 3′ section of HEP.

Also shown in FIG. 6, the first TFO 138 associated with the 3′ end 137 of the first strand AE-DNA 101 binds in a sequence specific manner to the targeted double stranded DNA 149 according to triplex binding rules well described by Hoogsteen. Similarly, the second TFO 139 associated with the 3′ end 141 of the second strand AE-DNA 102 also binds in a sequence specific manner to the target double DNA 149. Due to the positioning of the two TFOs 138, 139, the target DNA 149 is straddled by the bilateral AE-DNA 136, enabling the cleavage and replacement of a portion of the targeted duplex DNA with the HEP sequences 135, 155. When this reaction is complete, the base pair or series of base pairs of the first and second HEP sequences 135, 155 will be exchanged into the target DNA in a sequence specific manner. This system relies on the repair machinery within living cells to complete the digestion of the extraneous portions of DNA (i.e. the remnant TFO, EC components). The repair machinery also is necessary to cause the re-ligation of the free 5′ ends of the substrate DNA. These normal DNA repair mechanisms variably digest the 3′ end of the two ends of the bilateral AE-DNA 136 as well as the 5′ ends of the original target DNA 149 and re-ligate the ends when the digested portions match. The digestion would preferentially occur in a 3′ to 5′ direction since these are free single stranded DNA ends. By altering the length and/or the composition of the 3′ ends 137, 141 of the AE-DNA components, a change in the pattern of the in vivo repair function can be designed. This alteration may be predictable enough to tailor the length of the repair process of which the exchange reaction occurs.

An important aspect of the present invention is the ability to design the bilateral AE-DNA such that a variable number of nucleotide bases can be set between the 5′ reaction sites of the two AE-DNA strands. That is, the number of nucleotide bases present in the HEP sequence and exchangeable with the double stranded target DNA through the bilateral AE-DNA reaction can range from one to tens of thousands. Another aspect of this method is the ability to exchange DNA into the double stranded target DNA of any sequence composition of choice. Using this method, gene therapy could be accomplished by correcting a defective gene(s) (e.g. sickle cell anemia, cystic fibrosis, Huntington's chorea, etc.) or exchanging DNA sequences to cause gene dysfunction of an undesirable gene product.

III. Catalytic DNA Sequences That Facilitate DNA Cleavage and Re-Ligation Reactions

The reactions described hereinabove demonstrate the feasibility of the DNA self-exchange reaction and has been used to identify DNA sequences capable of promoting these reactions. The unilateral exchange reactions shown in FIGS. 1 and 2 are useful and in some embodiments are designed to select for DNA molecules capable of cleaving a receptor DNA (“substrate” or “target”). Moreover, the selected DNA molecules are capable of subsequently ligating a donor DNA (“HEP”) into the point of cleavage. This series of steps requires catalysis in order to proceed at an appreciable rate.

Sequences were sought and identified that exhibit the catalytic actions of promoting both cleavage of targeted DNA substrate as well as re-ligation with the exchanged DNA. These sequences, in fact, have catalytic activity under the experimental conditions provided that emulate physiological conditions that comprised an aqueous solution of 140 mM potassium chloride, 15 mM Hepes buffer at pH 7.4, 2 mM MgCl₂, and 1 mM spermine per 50 microliters of solution, although other reaction conditions are usable. The reaction conditions were also carried out at 37° C.

The following catalytic sequences were identified empirically by these techniques. The sequences are presented in symbolic form wherein “G” symbolizes a guanine nucleotide, “C” symbolizes a cytosine nucleotide, “A” symbolizes an adenine nucleotide, and “T” symbolizes a thymine nucleotide. The nucleotides are 15 nucleotides bonded covalently to one another in a linear fashion, although the number of nucleotides in this catalytic domain may be greater or fewer than this.

| | |
|---|---|
| 5′-GGA GCA TCA GTC TAT-3′ | (SEQ ID NO:5) |
| 5′-AAC CAG TCG GAG AGG-3′ | (SEQ ID NO:6) |
| 5′-CGG AGC ATC AGT CTA-3′ | (SEQ ID NO:7) |
| 5′-CAA AGT TTG GCT CCC-3′ | (SEQ ID NO:8) |
| 5′-CAC GTA CGC TGT CAC-3′ | (SEQ ID NO:9) |
| 5′-TGG TTG GTA AAA ATT-3′ | (SEQ ID NO:10) |
| 5′-GGC ACG CGG CGC T-3′ | (SEQ ID NO:20) |

In the presence of a divalent cation co-factor, such as magnesium, and so long as activated phosphorylated ligatable ends or other means of providing energy are present, these sequences perform the desired catalyzing functions without requiring the presence of ATP (adenosine 5′-triphosphate). However, in alternative embodiments ATP or another energy-generating compound is utilized to facilitate the reaction(s).

Besides the hereinabove disclosed N15 sequences, it is understood that other nucleotide sequences may exist possessing similar catalytic properties. Although in preferred embodiments the EC sequences are about 15 nucleotides, given that this number has been found to readily generate sequences capable of providing catalytic action, a skilled artisan recognizes herein that this number may be altered. Based on the teachings provided herein, a skilled artisan recognizes how to test for these sequences.

IV. Rotation of Elements Within the AE-DNA Sequences

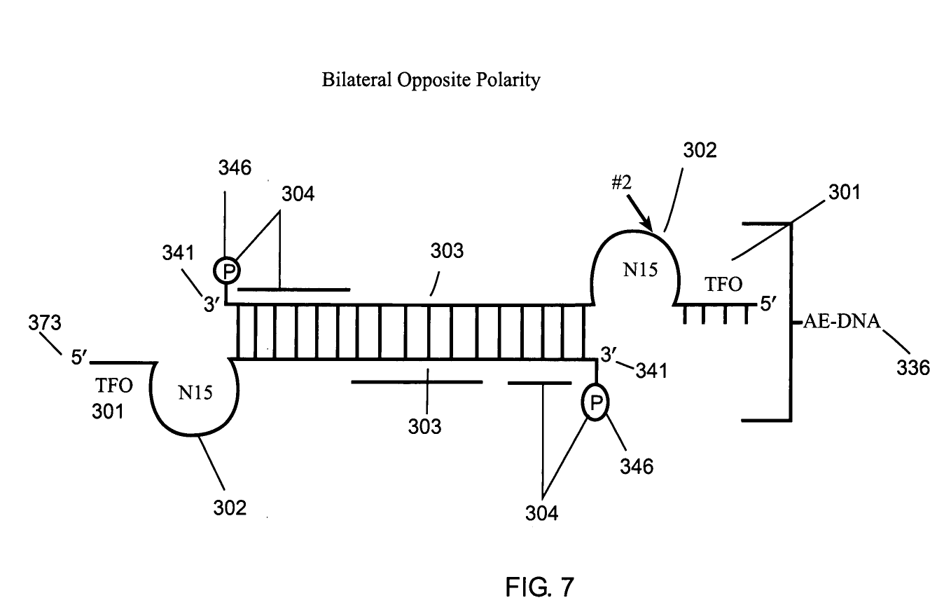

As illustrated in FIG. 7 and described in detail elsewhere herein, the four DNA sequence components of an AE-DNA (shown is an exemplary bilateral AE-DNA) are the DNA sequence recognition component (triplex forming oligonucleotide, or TFO) 301, the catalytic DNA enzyme component (EC) 302, the sequence of homology shared with the “target” DNA (homologous exchangeable piece, or HEP) 303, and the sequence that helps facilitate the exchange ligation portion of the reaction (activated ligatable end, or ALE) 304. The four components make up a basic structure capable of achieving both unilateral and bilateral DNA exchange reactions.

A skilled artisan recognizes that the four components may be ordered relative to each other in different arrangements to perform various tasks. The rearrangement of these components is designed to be responsive to the needs of an individual gene(s) system of interest. The arrangement is chosen in light of the system's sensitivity to arrangement variations with regard to the system's ability to accord an exchange of DNA sequences, and one of skill in the art knows how to do so based on the teachings provided herein.

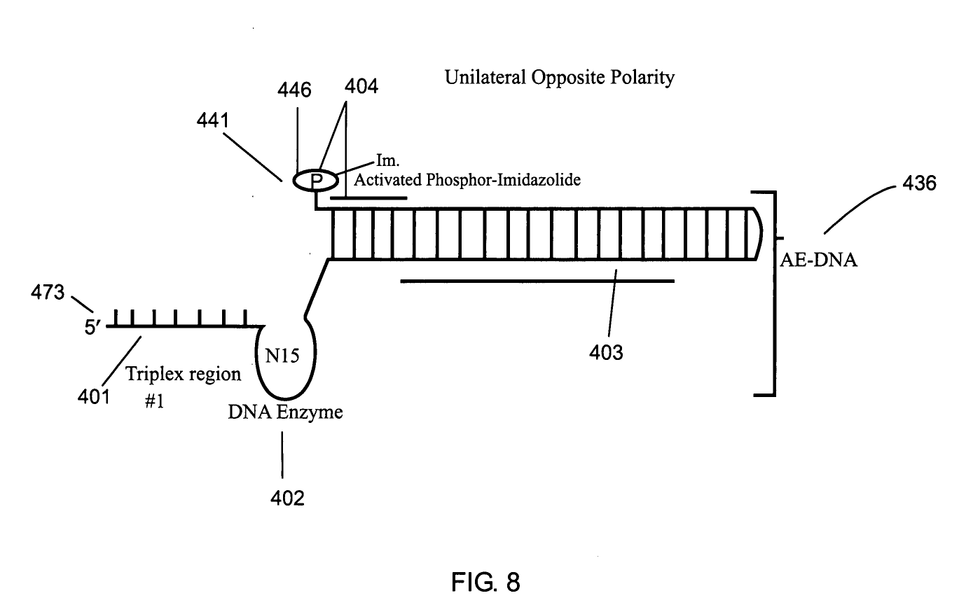

For example, referring now to FIGS. 7 and 8, it may be desirable to reorder the functional units in the respective HEP's to reverse the DNA strand's polarity. In contrast to the bilateral AE-DNA shown in FIG. 4, the bilateral AE-DNA 336 in FIG. 7 and the unilateral AE-DNA 436 of FIG. 8 are arranged such that the TFO sequences 301 and 401, respectively, are positioned proximal to the 5′ end 373, 473 of the respective AE-DNA strands 336, 436. Although similar to the AE-DNA 136 as originally described and shown in FIG. 4, this arrangement requires placing an activated phosphate group 346, 446, respectively, at the 3′ end 341, 441 rather than the 5′ end of the AE-DNA strands. With the ALEs 304 and 404 now situated in the 3′ position, reactions of the AE-DNA 336, 436 would now occur at the activated 3′ region. The EC sequence 302, 402 is still adjacent to the TFO and the HEP 303, 403 is still positioned between the EC and the ALE in this illustrative embodiment.

In some aspects of the invention, the chemical modification of the 3′ ends 341, 441 represents a less common solid state synthesis chemistry for synthetic oligonucleotides, which may offer advantages related both to stability of the AE-DNA in an in vivo environment and to the efficiency of the cellular repair function necessary to complete the DNA exchange reactions.

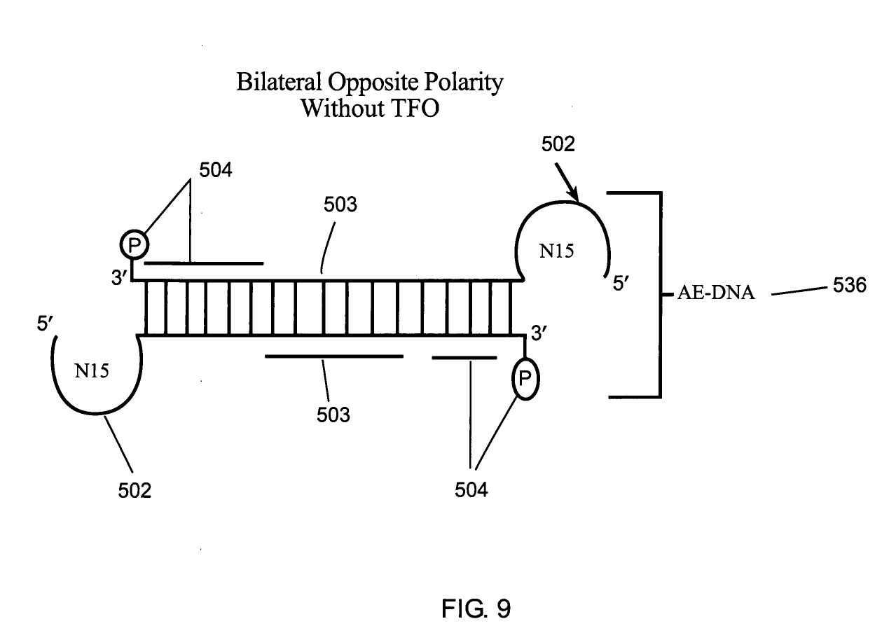

FIG. 9 illustrates another ordering of DNA functional unit sequences of a bilateral AE-DNA 536 including an EC sequence 502, and HEP sequence 503 and an ALE sequence 504 but lacking a TFO sequence. Because the TFO facilitates recognition of the target sequence along with a portion of homology conferred HEP, an AE-DNA lacking the TFO sequence may suffer reduced efficiency in recognizing the target sequence. In some embodiments, however, the loss in recognition efficiency may be outweighed by the benefit achieved in reducing the size of the AE-DNA.

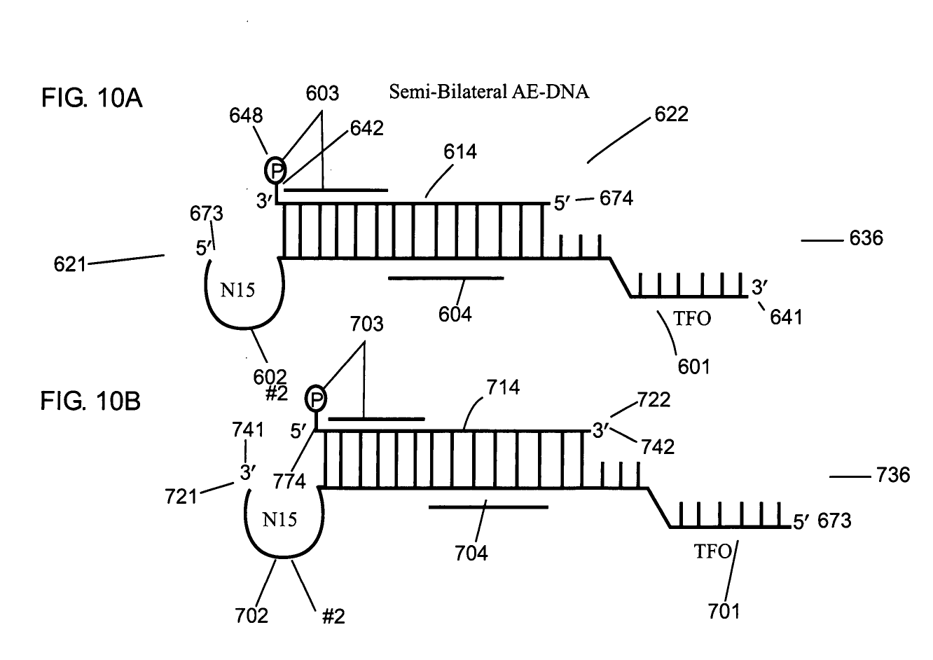

FIG. 10A shows another alteration of the DNA sequence components, wherein an AE-DNA 636 has a first strand 621 of nucleotides comprising in order from a 5′ end 673 to a 3′ end 641 an EC sequence 602, first HEP sequence 604 and a TFO sequence 601. The AE-DNA 636 has a second strand 622 comprising from a 5′ end 674 to a 3′ end 642, a second HEP sequence 614 and an ALE component 603 capped with an activated phosphate 648. In this arrangement, the first strand 621 also could lack a ligatable end and the EC 602 could be arranged with the TFO 601. This arrangement produces a “semi-bilateral” AE-DNA reaction wherein only the second strand 622 of DNA is available for exchange reactions with a target DNA since the first strand lacks an ALE. FIG. 10B shows a second semi-bilateral AE-DNA 736 having a reverse polarity from that of the first semi-bilateral AE-DNA 636 wherein similar numbers correspond to similar parts (i.e. second HEP sequence 614 of the first semi-bilateral AE-DNA 636 corresponds to a second HEP sequence 714 of the second semi-bilateral AE-DNA 736, and so forth).

Alternatively, a semi-bilateral AE-DNA duplex DNA can be arranged to comprise two regions of TFO sequences located on the same monofilament DNA strand. As with other semi-bilateral designs, this construct would have only one EC and only one ALE so that it would be capable of cleaving and re-ligating only a single strand of a double stranded target DNA. In some instances, the recognition sequences of the target DNA surrounding the point at which an exchange reaction is desired may provide a poor matching for the HEP. Since the localization of the point for exchange preferably occurs through triplex binding to the DNA target, the AE-DNA configured with plural TFO sequences could provide better matching ability and binding stability. In yet another variation, the semi-bilateral DNA may lack the HEP.

Those skilled in the art would recognize that there are numerous possible variations and arrangements of the four DNA sequence units that may be selected so as to promote desired DNA exchange reactions. A skilled artisan recognizes based on the teachings provided herein that the reactions between the AE-DNA (donor DNA) and a “target” (substrate or receptor DNA) can be promoted by either unilateral, bilateral or semi-bilateral reactions.

V. Alternative Activating Groups for the ALE

The fourth component of the AE-DNA termed the “Activated Ligatable End” or “ALE” has been described herein in some embodiments as a phosphor-imidazolide group. Ligation with a DNA molecule requires a phosphodiester linkage serving to anneal newly added sequences into the DNA's phosphodiester backbone. Such ligation requires, therefore, the inclusion of a phosphate component to complete this critical step that emulates the natural DNA replication process.

The imidazolide component of the phosphate group is an “activating” group capable of forming a covalent linkage (ligation process) when a free 3′ hydroxyl group is available on a target DNA strand after the cleavage of the phosphodiester backbone. The reactions of the AE-DNA utilize a phosphor-imidazolide group rather than the tri-phosphate residue present in the course of natural DNA replication. Under the natural process, a pyrophosphate group is hydrolyzed during the formation of a new phosphodiester covalent bond; in this sense the pyrophosphate can be considered as an activating group, indicating that a triphosphate group is interchangeable with the phosphor-imidazolide group of the activated ligatable end.

Those skilled in the art would recognize that, in addition to the phosphor-imidazolide group and a triphosphate group, other activating groups may be covalently linked to an alpha positioned phosphate group of the ALE. Preferably, these groups would be hydrolyzed while facilitating the formation of a covalent phosphodiester linkage when provided with an adequate receptor DNA molecule with a free 3′ hydroxyl group. These activating groups would ideally act as good leaving groups when nucleophilic attack is provided by the free 3′ hydroxyl group of the newly cleaved phosphodiester backbone of the target DNA strand. These activating groups may include, but are not limited to, other deoxyribonucleosides such as adenine, guanine, thymine, or cytosine. These bases may be linked to the alpha phosphate group via either a 3′ linkage or a 5′ linkage. The 3′ linkage may be utilized to reduce exonuclease activity in biologically applied systems.

Variable ribonucleoside bases also could be used as activating groups including, but not limited to, inositol and uracil. Also derivatives of imidazolide such as a 4,5-imidazoledicarboxylic acid or a 2-imidazolidonethione might serve as activating groups. With each substitution of the activated phosphate group, it is understood that the ALE thus produced may be incorporated into an AE-DNA molecule to facilitate the functionings and therapeutic advantages realizable therefrom in the process of replacing defective nucleotides in a target DNA. These activating groups represent a few of the many possible compounds that might be chosen to activate the phosphorylated groups of the ALE in lieu of the imidazolide group previously disclosed.

VI. Triplex Features

Triplex formation, also known as triplex strand formation, occurs when a DNA or RNA oligonucleotide selectively binds to a homopurine sequence region of a duplex DNA target. The small oligonucleotides that participate in triplex formation are also known as triplex-forming oligonucleotides (TFOs). These triplex binding oligonucleotides bind within the major groove of DNA forming what is known as Hoogsteen or reverse Hoogsteen hydrogen bonds within the purine-rich strand. A TFO can be broadly categorized as either having a pyrimidine or a purine-binding motif depending on its composition and orientation of binding relative to a duplex DNA target. Within the pyrimidine-binding motif, a TFO consisting of thymine (T) or cytosine (C) binds to the purine-rich strand of DNA in a parallel fashion via Hoogsteen bonds. In this case, the triplex binding of thymine (T) bases preferentially bind to the adenine (A) in an A:T base pair, and protonated cytosines (C+) bind to guanine (G) within G:C base pairs. The major complication with pyrimidine motif binding is that it requires acidic condition so that the N3 of the cytosine (C+) base is protonated. It usually renders the TFO unable to bind with high affinity at physiological pH.

In the purine motif binding, the third strand binding again occurs within the major groove and binds to homopurine tracks with adenine (A) binding to an A:T base pair. Similarly, the guanine (G) binds specifically to G:C base pair. Additionally, purine motif allows triplex binding of thymine (T) to A:T base pairs with slightly lower binding efficiency. In contrast to pyrimidine binding, the purine TFO binds in an anti-parallel fashion to the purine-rich strand in the duplex DNA target. This binding of the purine motif binds to duplex DNA within the major groove via reverse Hoogsteen bonds. Additionally, since protonation is not required, the purine motif TFO binding is largely independent of pH. Since small inversions within homopurine tracks can disrupt triplex binding (inversions such as T:A or C:G), a number of alternative base modifications or unnaturally occurring bases can be incorporated into a TFO to overcome some of the imperfect sequences that would be found within genes that are targets for genetic therapy. Specifically, in the case of purine motif binding, in addition to adenine (A) binding to an A:T base pair target it has been found that thymine (T) can also bind in a triplex manner efficiently (Beal and Dervan, 1991). Additionally, 7-Deaza-2-deoxyzanthosine (a purine analogue) can also bind to this A:T base pair (Olivas and Maher, 1995). Also, within the purine binding motif the base pair target of G:C normally binds guanine (G), however this can also bind effectively with 2-Deoxy-6-thioguanosine. This substitution can overcome some of the inhibition that is seen by high potassium levels or monovalent cations in general. (Olivas and Maher, 1995). Additionally, the C:G inversion can perform moderate binding with thymine (T). The modified base 5-fluoro-deoxyuracil can also bind to the inversion base pair relatively strongly (Durland et al., 1994), with pyrimidine motif TFO binding alternate or modified bases that can bind to standard triplex structures as well as with T:A in a weak fashion by guanine. Additionally, a modified base compound known as D3 (also known as 1-(2-deoxy-b-D-ribofuranosyl)-4-(3-benzanidophenyl)imidazole) was found not only to bind T:A base pair inversions but also all four base pair combinations within the pyrimidine motif. Within the pyrimidine-binding motif the C:G base pair inversion may also be bound in a weak fashion by thymine or cytosine.

As can be seen by one skilled in the art, multiple changes with nucleoside base modifications can be used to adequately target an imperfect homopurine duplex DNA target site. Also, those skilled in the art will recognize that a number of other modifications, such as changing the charge of the natural phospodiesterase backbone, can be used to relieve interfering negative charging, which has a tendency to cause dimers and tetramers with very G-rich oligonucleotides. These formed dimers and tetramers interfere with the ability of these TFOs to participate in triplex binding. One method to reduce the inhibition seen occasionally with monovalent cations, particularly potassium, is to use N,N-diethylethylenediamine phosphoramidate linkages that contain both primary and tertiary amine groups. These positive amine groups, when replaced with the natural phospodiesterase bond, are quite effective at reducing or eliminating the negative effect on monovalent cations. Lastly, those skilled in the art would also recognize that tethering strategies by using linker molecules such as successive carbon polymers can potentially bind to nearby homopurine tracks that may not be accessible to a continuous TFO sequence. However, it should be recognized that many of these alterations add to the general ability to the triplex forming oligo strategies as a method to target the composite AE-DNA structures to gene sequence of interest. These targeting strategies of incorporating TFO as part of the AE-DNA constructs provide a convenient and versatile method to place the catalytic portion of the AE-DNA in proper position to carry out single stranded nucleic acid AE-DNA into a duplex DNA gene target by a transesterification reaction.

VII. Nucleic Acids

The term “nucleic acid” or “polynucleotide” will generally refer to at least one molecule or strand of DNA, RNA or a derivative or analog thereof, comprising at least one nucleobase, such as, for example, a naturally occurring purine or pyrimidine base found in DNA (e.g. adenine “A,” guanine “G,” thymine “T” and cytosine “C”) or RNA (e.g. A, G, uracil “U” and C). The term “nucleic acid” encompasses the terms “oligonucleotide” and “polynucleotide.” The term “oligonucleotide” refers to at least one molecule of between about 3 and about 100 nucleobases in length. The term “polynucleotide” refers to at least one molecule of greater than about 100 nucleobases in length. These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss”, a double stranded nucleic acid by the prefix “ds”, and a triple stranded nucleic acid by the prefix “ts.”

Nucleic acid(s) that are “complementary” or “complement(s)” are those that are capable of base-pairing according to the standard Watson-Crick, Hoogsteen or reverse Hoogsteen binding complementarity rules. However, in a specific embodiment, a primer of the present invention comprises a majority of nucleotides that are incapable of forming standard Watson-Crick base pairs, particularly with other nucleotides within the same primer.

As used herein, the term “complementary” or “complement(s)” also refers to nucleic acid(s) that are substantially complementary, as may be assessed by the same nucleotide comparison set forth above. The term “substantially complementary” refers to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double stranded nucleic acid molecule during hybridization. In certain embodiments, the term “substantially complementary” refers to at least one nucleic acid that may hybridize to at least one nucleic acid strand or duplex in stringent conditions. In certain embodiments, a “partly complementary” nucleic acid comprises at least one sequence that may hybridize in low stringency conditions to at least one single or double stranded nucleic acid, or contains at least one sequence in which less than about 70% of the nucleobase sequence is capable of base-pairing with at least one single or double stranded nucleic acid molecule during hybridization.

As used herein, “hybridization”, “hybridizes” or “capable of hybridizing” is understood to mean the forming, of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating at least one nucleic acid, such as a gene or nucleic acid segment thereof, or detecting at least one specific mRNA transcript or nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence of formamide, tetramethylammonium chloride or other solvent(s) in the hybridization mixture. It is generally appreciated that conditions may be rendered more stringent, such as, for example, the addition of increasing amounts of formamide.

It is also understood that these ranges, compositions and conditions for hybridization are mentioned by way of non-limiting example only, and that the desired stringency for a particular hybridization reaction is often determined empirically by comparison to one or more positive or negative controls. Depending on the application envisioned, it is preferred to employ varying conditions of hybridization to achieve varying degrees of selectivity of the nucleic acid(s) towards target sequence(s). In a non-limiting example, identification or isolation of related target nucleic acid(s) that do not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

As used herein a “nucleobase” refers to a naturally occurring heterocyclic base, such as A, T, G, C or U (“naturally occurring nucleobase(s)”), found in at least one naturally occurring nucleic acid (i.e. DNA and RNA), and their naturally or non-naturally occurring derivatives and analog. Non-limiting examples of nucleobases include purines and pyrimidines, as well as derivatives and analog thereof, which generally can form one or more hydrogen bonds (“anneal” or “hybridize”) with at least one naturally occurring nucleobase in manner that may substitute for naturally occurring nucleobase pairing (e.g. the hydrogen bonding between A and T, G and C, and A and U).

As used herein, a “nucleotide” refers to a nucleoside further comprising a “backbone moiety” generally used for the covalent attachment of one or more nucleotides to another molecule or to each other to form one or more nucleic acids. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when the nucleotide comprises derivatives or mimics of a naturally occurring 5-carbon sugar or phosphorus moiety, and non-limiting examples are described herein.

VIII. Pharmaceutical Compositions and Routes of Administration

Compositions of the present invention will have an effective amount of a nucleotide sequence for therapeutic administration in combination and, in some embodiments, is combined with an effective amount of a compound (second agent) that is therapeutic for the respective appropriate disease or medical condition. Such compositions will generally be dissolved or dispersed in a pharmaceutically acceptable carrier or aqueous medium. The term “effective” as used herein refers to inhibiting an exacerbation in symptoms, preventing onset of a disease, amelioration of at least one symptom, or a combination thereof, and so forth.

The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-disease agents, can also be incorporated into the compositions.

In addition to the compounds formulated for parenteral administration, such as intravenous or intramuscular injection, other pharmaceutically acceptable forms include, e.g., tablets or other solids for oral administration; time release capsules; and any other form currently used, including cremes, lotions, mouthwashes, inhalants and the like.

The compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The compositions of the present invention may advantageously be administered in the form of injectable compositions either as liquid solutions or suspensions; solid forms suitable for solution in, or suspension in, liquid prior to injection also may be prepared. These preparations also may be emulsified. A typical composition for such purposes comprises a 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters, such as theyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

All of the essential materials and reagents required for therapy may be assembled together in a kit. When the components of the kit are provided in one or more liquid solutions, the liquid solution preferably is an aqueous solution, with a sterile aqueous solution being particularly preferred.

For in vivo use, an agent may be formulated into a single or separate pharmaceutically acceptable syringeable composition. In this case, the container means may itself be an inhalant, syringe, pipette, eye dropper, or other such like apparatus, from which the formulation may be applied to an infected area of the body, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the gene therapy and/or the chemotherapeutic drug.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

The active compounds of the present invention will often be formulated for parenteral administration, e.g., formulated for injection via the intravenous, intramuscular, subcutaneous, or even intraperitoneal routes. The preparation of an aqueous composition that contains a second agent(s) as active ingredients will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the therapeutic formulations of the invention could also be prepared in forms suitable for topical administration, such as in cremes and lotions. These forms may be used for treating skin-associated diseases, such as various sarcomas.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In one exemplary method wherein the therapy is for an individual with cancer, targeting of cancerous tissues may be accomplished in any one of a variety of ways. For example, a composition of the present invention, such as an AE-DNA with an appropriate homologous exchangeable piece may be complexed with liposomes and injected into patients with cancer; intravenous injection can be used to direct the gene to all cell. Directly injecting the liposome complex into the proximity of a cancer can also provide for targeting of the complex with some forms of cancer. For example, cancers of the ovary can be targeted by injecting the liposome mixture directly into the peritoneal cavity of patients with ovarian cancer. Of course, the potential for liposomes that are selectively taken up by a population of cancerous cells exists, and such liposomes will also be useful for targeting the gene.

Those of skill in the art will recognize that the best treatment regimens for using a composition of the present invention to provide therapy can be straightforwardly determined. This is not a question of experimentation, but rather one of optimization, which is routinely conducted in the medical arts. For example, in vivo studies in nude mice provide a starting point from which to begin to optimize the dosage and delivery regimes. The frequency of injection will initially be once a wk, as was done some mice studies. However, this frequency might be optimally adjusted from one day to every two weeks to monthly, depending upon the results obtained from the initial clinical trials and the needs of a particular patient. Human dosage amounts can initially be determined by extrapolating from the amount of composition used in mice. In certain embodiments it is envisioned that the dosage may vary from between about 1 mg composition DNA/Kg body weight to about 5000 mg composition DNA/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg composition DNA/Kg body to about 20 mg composition DNA/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

IX. Delivery of Nucleic Acid Molecules

Several non-viral gene delivery vectors for the transfer of a polynucleotide(s) of the present invention into mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub; 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al, 1990), and receptor-mediated transfection (Wu and Wu, 1987; Wu and Wu, 1988). Some of these techniques may be successfully adapted for in vivo or ex vivo use.

Once the polynucleotide has been delivered into the cell the sequence of the AE-DNA homologous to the target DNA is positioned accordingly. In certain embodiments, the polynucleotide may be stably integrated into the genome of the cell by methods described herein. How the polynucleotide is delivered to a cell and where in the cell the nucleic acid remains is dependent on a number of factors known in the art.

In yet another embodiment of the invention, the expression vector may simply consist of naked recombinant DNA or plasmids comprising the polynucleotide. Transfer of the construct may be performed by any of the methods mentioned above which physically or chemically permeabilize the cell membrane. This is particularly applicable for transfer-in vitro, but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention, transfer of a naked DNA into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, a polynucleotide may be entrapped in a liposome, another non-viral gene delivery vector. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the liposome may be complexed with a hemagglutinatin virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other polynucleotides that can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0 273 085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells.

In certain embodiments, DNA transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Unilateral AE-DNA Exchange Reaction and Identification of Catalytic Sequences

The reaction steps outlined hereinbelow illustrate a specific embodiment for the generalized scheme described herein. In specific embodiments it is used to detect nucleic acid catalytic sequences capable of performing both sequence specific cleavage and re-ligation reactions. This methodology also can be used to improve upon various discovered catalytic sequences.

The reaction was performed starting with two synthesized oligonucleotides:

1. Oligonucleotide #1 Biotin linked 5=DNA target (or “substrate”) is referred to as Subst-enz-101 and comprises the nucleotide sequence:

(SEQ ID NO:1)

5′ GCG TTC GGC AAC ACT TTC TCT TAT GGT GTT CAA GCA
CTT GTC AAG TGC TTG AAC ACC ATA AGA GAA AG-3′

Oligonucleotide #2 monofilament “activated Exchangeable DNA” (AE-DNA) with 5′ phosphor-imidazolide is referred to as N15-enz-101 and comprises the nucleotide sequence:

(SEQ ID NO:2)

5′-(P-Im)-TGT TCA AG ATC GGG TCT GCA G ATC TCC GGA
TAC NNN NNN NNN NNN NNN TTC TCT TTC TAC ACT GGC
TAG CAA C-3′

wherein “N” represents random nucleotides selected from A, T, C, G, or analogs thereof.

A monofilament nucleic acid sequence is utilized, comprising a first sequence capable of forming triplex forming oligonucleotide interactions with a target DNA sequence according to Hoogsteen binding rules, further comprising a second sequence capable of catalyzing a sequence specific cleavage reaction through the hydrolosis of a phosphodiester linkage in the target DNA sequence, wherein the second sequence also is capable of catalyzing ligation reactions in a sequence specific manner to form ligative bonds between a first and a second strand of DNA by formation of a phosphodiester covalent bond between two nucleotides bases, each of the first and second strands of DNA having a 3′ end and a 5′ end, further comprising a third sequence that is homologous with the target DNA sequence for up to 100,000 DNA nucleotide bases, wherein the second sequence has a 5′ end and a 3′ end and may be identified by the sequence: 5′-GGA GCA TCA GTC TAT-3′ (SEQ ID NO:5); 5′-TGG TTG GTA AAA ATT-3′ (SEQ ID NO:10); 5′-AAC CAG TCG GAG AGG-3′ (SEQ ID NO:6); or 5′-CGG AGC ATC AGT CTA-3′ (SEQ ID NO:7); 5′-CAA AGT TTG GCT CCC-3′ (SEQ ID NO:8); 5′-CAC GTA CGC TGT CAC-3′ (SEQ ID NO:9); 5′-GGC ACG CGG CGC T-3′ (SEQ ID NO:20).

The phosphor-imidazole group was added to the 5′ terminal phosphate of the N15-enz-101 oligonucleotide by combining imidazole at a concentration of 0.1 M (pH 6.0) to 1 microgram of oligo, adding 1-[3-dimethylaminopropyl]-3-ethyl carbodiimide to a final concentration of 0.15 M, incubating at room temperature (25° C.) for 1 hour and then neutralizing with 10 mM Hepes (pH 7.2). Salts were removed from the reaction mixture using a G25 Sephadex column by standard means in the art.

The DNA exchange reaction was performed by first immobilizing the target Subst-enz 101 (biotin-labeled oligo) to a magnetic bead slurry labeled with streptavidin. Various concentrations were used ranging from 50 to 500 ng immobilized and equilibrated with 15 mM Hepes (pH 7.4). The beads were suspended in 50 microliters of buffer containing 15 mM Hepes (pH 7.4), KCl at 140 mM, 2 mM MgCl₂, and spermine at 1 mM final volume. To this 500 ng AE-DNA (N15-enz-101) was added and incubated at 37° C. for one hour. After incubating, the beads were washed 10 times with 1 mL of NaCl buffer solution having a stringency increasing from 50 mM to 1M NaCl with 1 mM EDTA. A final wash was made using 0.1 N NaOH and then Tris HCl 10 mM with 1 mM EDTA (pH 7.4) to neutralize.

The beads were aliquoted at various wash stages and then subjected to PCR with primers complementary to only one or the other of the target (Subst-enz-101) or AE-DNA (N15-enz-101) starting oligonucleotides. The 15-cycle PCR revealed the expected 100 base pair product from two of the samples. These 100 base pair fragments were cloned by a TA cloning vector system (Invitrogen Corp.) following the manufacturer's instructions. Twenty clones were picked, with eight showing an insert. Sequencing of these clones demonstrated unique catalytic sequences and an exchange/splice junction at the predicted location.

### Example 2

### Bilateral AE-DNA Exchange Reaction

The bilateral AE-DNA reaction took advantage of the catalytic sequence data obtained in the unilateral AE-DNA reaction described above. This sequence information was used to design the two catalytic sequence components (the EC sequences) for the two DNA monofilaments that together form the bilateral AE-DNA.

The following oligonucleotides were synthesized and were designed to hybridize with each other to form the bilateral AE-DNA depicted in FIGS. 4 and 5.

(SEQ ID NO:3) Oligo #1 (first AE-DNA strand 201): 5′-(P-Im)-CAA GAT GGA TTG CAC GCA GGT TCT CTG ACT GCA ACC AGT CGG AGA GGC CCA CCT CTC C-3′

(SEQ ID NO:4) Oligo #2 (second AE-DNA strand 202): 5′-(P-Im)-CGG CCG GAG AAC CTG CGT GCA ATC CGT TTC GTC GGA GCA TCA GTC TAT TAG TAC CGT TTG CT-3′

The oligonucleotides were synthesized with 5′ phosphorylation. The activating imidazolide group was added to the 5′ end as a post-synthesis modification by standard means in the art. The procedure used was identical to that of the oligonucleotides used for the unilateral reaction. Specifically, the phosphor-imidazole group was added to the 5′ terminal phosphate of both synthesized oligonucleotides in separate reactions by combining imidazole at a concentration of 0.1 M (pH 6.0) to 1 microgram of each oligonucleotide. Thereafter, 1-[3-dimethylaminolpropyl]-3-ethyl carbodiimide was added to a final concentration of 0.15 M. The formulations were incubated at room temperature for one hour and then were neutralized with 10 mM Hepes (pH 7.2). The oligonucleotides then were desalted on a G25 Sephadex column by standard procedures.

Nucleotide sequences for enzymatic DNA components (EC) that promote the relevant cleavage and re-ligation reactions are described herein. These two DNA “enzymes” were discovered to catalyze these reactions under the experimental conditions provided by the unilateral reaction and have been used in a similar manner in the bilateral reaction.

To further demonstrate the utility of this bilateral exchange reaction described above, a plasmid comprising two antibiotic resistance genes relevant to ampicillin and kanamycin was selected as a suitable target DNA. The kanamycin relevant gene was rendered defective by engineering a single base substitution to create a stop codon positioned at a premature site, such as at amino acid position 8. The pair of experimental oligonucleotides comprised within the HEP region comprise a base pair that would correct the kanamycin resistance gene at amino acid position 8 if DNA exchange reactions occurred by and between the AE-DNA fabricated from the oligonucleotides and the kanamycin resistance gene.

The two oligonucleotides were annealed at 65° C. and were cooled slowly to room temperature over a period of time of approximately 15-20 minutes. The annealing reaction between the respective complementary HEP sequences 235, 255 produced the functional AE-DNA unit, and is illustrated in FIG. 5. The plasmids were suspended in 50 microliters of buffer containing 15 mM Hepes (pH 7.4), KCl at 140 mM, 2 mM MgCl₂, and spermine at 1 mM final volumes. The target plasmids were present in the 50 microliters in amounts ranging from 200 nanograms to one microgram by weight of DNA. To solution, 500 ng of the bilateral AE-DNA was added and allowed to incubate for one hour at 37° C. Appropriate positive and negative plasmid controls were also treated analogously.

After the incubation period during which the target plasmids and the bilateral AE-DNA were permitted to react with one another, the solutions were placed on ice. Two (2) microliters of the reaction solution were aliquoted and added to 50 microliters of competent E. coli cells (XL1-Blue; Stratagene). The competent cells were then transformed according to the manufacturer's directions. These cells were then plated on substrate containing both ampicillin and kanamycin. Clones that were resistant to both ampicillin and kanamycin represented approximately 10% that of the positive control plates (negative plates contained no growth). Clones were picked/sequenced and demonstrated correction of the mutated base pair at the correct site. The negative controls did not show any reverse mutants.

These results indicated correct functioning of the bilateral AE-DNA construct for this correction of a dysfunctional kanamycin resistance gene comprising one point mutation. The concept of sequence specific DNA strand exchange by this reaction was demonstrated by these experiments as an example of DNA base pair substitution.

### Example 3

### Therapeutic Applications

The methods and compositions provided herein are useful for providing therapy for a medical condition, wherein the condition comprises a defective nucleotide sequence that, in some embodiments, affects the activity or expression of a gene product. The present invention provides therapy by replacing at least the defective nucleotide sequence.

A medical care provider provides therapy to an individual afflicted with the medical condition comprising the defective nucleotide sequence by exchanging the defective sequence with a nondefective sequence or by reducing the levels or activity of an endogenous but deleterious gene product, such as an oncogene. The care provider identifies the therapy to be remedied and determines the appropriate nucleotide sequence in the database for the therapy, by standard means in the art and the known literature. The sequence for the methods and compositions for utilization preferably derive from the same organism as the one being treated, although interspecies sequences are utilized in some embodiments, so long as a similar function for the sequence is maintained.

Following identification of the appropriate sequence, it is obtained, such as by polymerase chain reaction methods well known in the art. Following polymerase chain reaction, in some embodiments the nucleotide sequence obtained is sequenced to verify the accuracy of the sequence. The skilled artisan then utilizes the sequences in methods and compositions described herein.

### Example 4

### Screening for Catalytic Nucleic Acid Sequences Capable of Transesterification

The following is an exemplary procedure that allows for an efficient means to screen for catalytic nucleic acid sequences that are capable of performing transesterification. This screening tool can considered a “panning” tool to rapidly screen for catalytic single stranded nucleic acid sequences, either DNA, RNA, or a DNA/RNA chimera, that can function or assist in the catalysis of transesterification reactions.

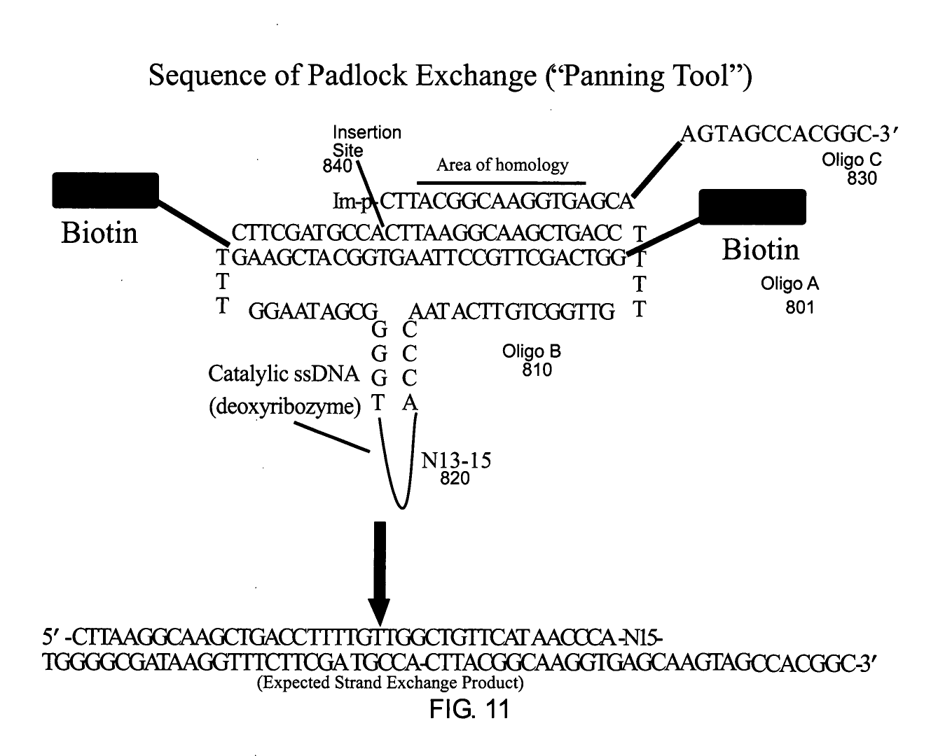

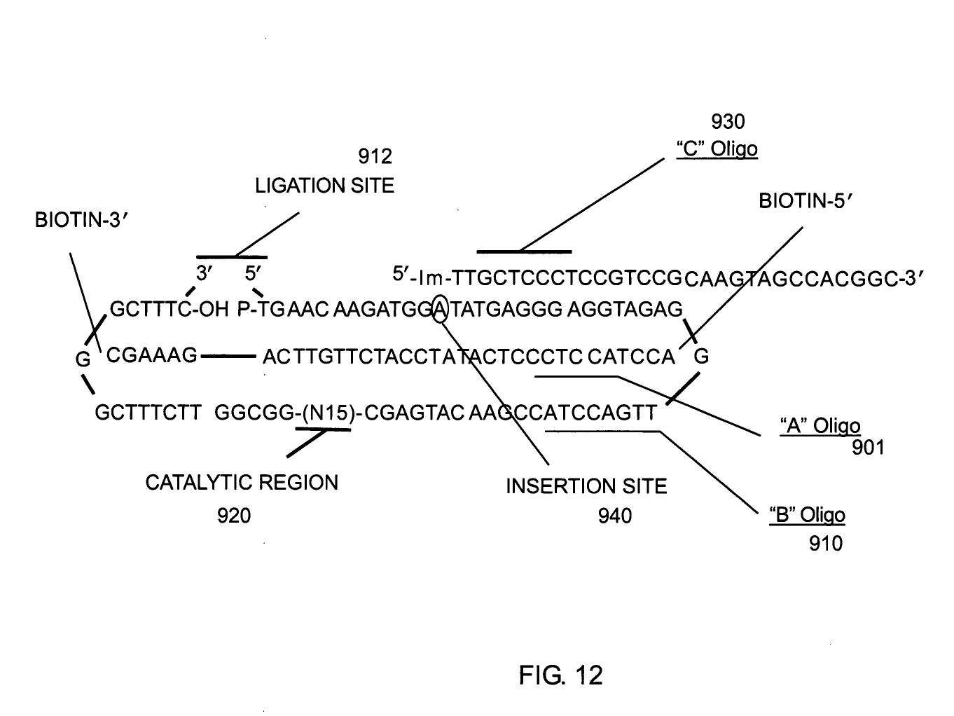

The system illustrated in FIGS. 11 and 12 is one embodiment of assaying for an enzymatic domain, wherein the system comprises three synthetic oligos, with exemplary embodiments having different representative sequences as illustrated in each of FIGS. 11 and 12. One oligo is bis-biotinylated at both the 3′ and 5′ ends (oligo A; 801, 901, respectively) as one exemplary means for immobilization when desired. The second oligo (oligo B; 810, 910, respectively) is cyclized (in FIG. 12, at the region termed “ligations site” 912) and comprises the exemplary 15-nucleotide enzymatic insert (catalytic region) 820, 920, respectively, although the size of the enzymatic insert may be greater or fewer in the number of nucleotides. The third oligo has a phosphor-imidazolide at the 5′ end (oligo C; 830, 930, respectively). Initially, oligo A 801, 901 is allowed to hybridize to the oligo B 810, 910 and the complex is subsequently bound to a streptavidin column. After immobilization, oligo B 810, 910 is unable to be denatured from the column since it is wound around oligo A 801, 901. Therefore, no cyclic oligos can be washed from the column unless cleavage occurs to open the macrocyclic oligo. In specific embodiments, the conditions are such that the enzymatic region is in close proximity to the site of desired cleavage and insertion of the phosphor-imidazolide oligo; in exemplary embodiments illustrated as 840, 940, respectively. The phosphor-imidazolide-containing oligo C 830, 930 has been designed to form a triplex structure with the simulated duplex DNA structure (i.e., oligos A 801, 901 and B 810, 910). If cleavage and re-ligation occurs between oligo B 810, 910 and oligo C 830, 930, a new oligo product would be formed and would be recoverable from the column under denaturing conditions. In FIG. 11, an expected strand exchange product 850 is illustrated. Also, for example, in FIG. 12 this product would have a length of 115 bases since the cyclic oligo originally contained 85 bases and the phosphor-imidazolide oligo contained 30 bases. Multiple new 115 base oligos have been isolated after first round selection. These 115-mer products have been cloned and sequenced and contain the predicted insertion of nucleotides at the correct location.

Thus, in general exemplary embodiments of this procedure, four components of AE-DNA are incorporated into the three oligos (A, B, and C), such that after successful DNA exchange reactions, products are easily isolated as column elutant. In brief, the procedures of these reactions are as follows. The DNA exchange reaction is performed by first hybridizing oligos A and B together and then immobilizing this annealed product to a streptavidin column in 15 mM Hepes, pH 7.4. Various concentrations are utilized, ranging from 50 to 500 ng of duplex DNA. Oligo C is pre-activated at the 5′ phosphate by adding a mixture of imidazole and 1-(3-dimethylaminopropyl)-3-ethylcarbodiimide at a concentration of 0.1 M (pH 6.0) to 1 μg of oligo, by the procedure of Chu et al. (1983). A buffer containing 15 mM Hepes (pH 7.4), KCl at 140 mM, 2 mM MgCl₂, and spermine combined with activated oligo C is then added to the column. After incubating for 1 hr at 37° C., the column is washed 10 times with 1 ml of NaCl buffer solution having a stringency increasing from 50 mM to 1 M NaCl with 1 mM EDTA. A final wash is made using 0.1 NaOH and then Tris HCl 10 mM with 1 mM EDTA (pH 7.4) to neutralize. The various fractions are analyzed for 115-mer products and undergo PCR amplification for cloning and to generate a new pool of oligos to participate in subsequent selection rounds.

Thus, the following specific steps illustrate an exemplary means to screen for catalytic nucleic acid sequences.

Step 1

First, a closed circle single-stranded nucleic acid structure is made. This is performed after ligation of an oligonucleotide referred to as a “new-pad oligo” (in the embodiment described herein regarding GFP target sequence, an exemplary new-pad oligo is 5′-Phosphate-GCC ACT TAA GGC AAG CTG ACC TTTT GTT GGC TGT TCA TAA CCC A-N13-T GGG GCG ATA AGG TTT CTT CGA T-3′; SEQ ID NO:28) with a bridging oligonucleotide linker referred to as a “bridge-linker one” (an example of which is 5′-AAG TGG CAT CGA AG-3′; SEQ ID NO:24). These oligonucleotides are mixed at ratios of 1:1, 2:1 and 1:2 for a total concentration of 5 micrograms; this mixture is heated to 90° C. and slowly cooled to room temperature. When room temperature is reached, T4 ligase is added along with ligase reaction buffer, ATP and ligase enzymes, and the mixture is incubated at 37° C. for 2 hours. This procedure is repeated one additional time and then digested with exonuclease T (New England Biolabs) for one hour and denatured at 70° C. This is followed with phenol/chloroform extraction, and the closed circular nucleic acid structure is precipitated. This closed circle is referred to as circle-new-pad oligo.

Step 2

The bilateral-biotin-padlock oligonucleotide (an example of which is 5′-Biotin-TTT TGG TCA GCT TGC CTT AAG TGG CAT CGA AGT TTT-Biotin-3′; SEQ ID NO:26) is annealed with the circle-new-pad oligonucleotide at a ratio of 16-millimolar bilateral-biotin-padlock to 8-millimolar circle-new-pad oligo. The total volume of water is brought to 500 microliters, heated to 80° C. and slowly cooled to room temperature. This annealing reaction allows the bilateral biotin padlock to complementary bind with the single strand closed circular oligonucleotide prepared above, and after this is completed, this mixture will be added to an agarose-streptavidin column. Adding this mixture to the streptavidin column with binding of both the three-prime and five-prime ends of the padlock oligonucleotide effectively covalently links the closed circular single stranded nucleic acid molecule to the immobilized agarose beads within the column. This has the effect of rendering the closed circular nucleic acid structure unable to be denatured away from the bilateral-biotin-padlock oligonucleotide, without a cleavage of a covalent bond either within the padlock oligo or the closed circular single stranded nucleic acid structure.

Step 3

A number of columns are set-up containing agarose-linked streptavidin (capable of binding biotin) at 300 to 600 microliters in each mini column. These columns are washed 3 times with 300 microliters of PBS.

Step 4

The annealed oligo (circular new-pad oligo biotin-padlock) is added to the columns slowly. The column is washed with 300 microliters of PBS for 3 separate times and washed/denatured with 0.1 N NaOH, freshly made, at 100 microliters for 2 washes. The column is then washed again with 300 microliters PBS, three times again, and the wash is collected for control PCR testing.

Step 5

A freshly labeled imidazoline-labeled oligonucleotide is prepared, although in some embodiments this oligonucleotide is previously labeled. To phospho-imidazolide label oligonucleotides, such as 5′-phospho-oligonucleotides, obtain 100 micromolar at 1 microgram per micoliter of 5′-phospho-oligonucleotide (such as a 30-mer) (Total 20 micrograms per labeling). Imidazolide is added to a final concentration 0.1 Molar (pH 6.0). Fifteen microliters of 1 Molar 1-[3-(DIMETHYLAMINO)PROPYL]-3-ETHYLCARBODIIMIDE-HCl, final concentration 150 mM is added, to a total volume of 100 microliters and then incubated at room temperature for 1 hour. To this 1/10 volume of Na Acetate 3 Molar (pH 7.8) is added, and the nucleic acid is precipitated with 2.5× volumes of ice cold 100% ethanol. The solution is then microfuged at 4° C. at 14,000 RPM for 15 minutes, and the pellet is washed with 70% ethanol and dried. An example of a phospho-imidazolide oligonucleotide is 5′-Phosphate-CTT ACG GCA AGC TGA GCA AGT AGC CAC GGC-3′ (SEQ ID NO:27).

Step 6

A number of columns are made with various buffer concentrations and incubated with freshly prepared phospho-imidazolide oligonucleotide; however, in many embodiments all buffers will comprise KCl at 150 millimolar, 0.5 mM ATP, Hepes buffer (pH 7.4) at 20 mM and spermine at 2.5 mM. Various concentrations of MgCl₂ are also added, in some embodiments. In other embodiments, various concentrations of Zn²⁺, Ca²⁺, Mn²⁺, or histidine are added (with the concentration of the divalent cations ranging from about 1 mM to about 20 mM). In many preferred embodiments the buffers will also contain spermine at 2.5 millimolar, as well as Hepes buffer (pH 7.4) at 20 millimolar. All divalent cations range from a concentration of 1 millimolar to 20 millimolar concentration). In a specific embodiment, two micrograms of the phospho-imidazolide oligonucleotide are added.

Step 7

The various buffers are added to separate columns (already comprising the phospho-imidazolide oligonucleotide) and the column is clamped. The buffer is held for about 30 minutes to one hour at room temperature. The elution buffer is reapplied on the top of the column after it is drained by gravity and clamped again for 30 minutes to one hour at room temperature. After this, 300 microliters of 0.1 NaOH are added. Next, the elution buffer sample is collected and neutralized with 80 microliters of 0.5 NaOH and 40 microliters of 3M sodium acetate (pH 7.4); glycogen is used as a carrier for precipitation prior to the addition of ethanol.

Step 8

The precipitated material is resuspended in 20 microliters of water and checked for the expected strand exchange product by using polymerase chain reaction. Exemplary primers include primers 1 (5′-TTG TTG GCT GTT CAT AAC C-3′; SEQ ID NO:16) and primer 2 (5′-Phosphorylated-GCC GTG GCT ACT TGC T-3′; SEQ ID NO:17). PCR is performed of the various eluted fractions for 30 cycles at a PCR program as follows: five minutes of denaturation at 94° C. for one cycle, then 94° C. for one minute followed by an annealing temperature of 54° C. X 45 seconds, followed by an extension phase of 72° C.×45 seconds. These steps are repeated for 30 cycles and then completed with one 7-minute 72° C. extension for 1 cycle. The presence of the expected 93 base pair PCR fragment is checked on an 8% DNA acrylamide gel.

Step 9

The regeneration of a single-stranded nucleic acid fragment is performed, in exemplary embodiments by using primer #3 (5′-Biotin-GGT CAG CTT GCC TTA AGT GG-3′; SEQ ID NO:18) that has a 5-prime biotin link and primer # 4 (5′-Phosphorylated-TTTT GTT GGC TG TTC ATA ACC-3′; SEQ ID NO:19) that is used to amplify a new 81-base pair fragment that can then be reused after it is converted again to a closed single stranded nucleic acid structure. The pooled fragments 81-based pair nucleic acid fragments are then bridged together with a second plasmid, referred to as bridge-link-2 (an exemplary embodiment of which is SEQ ID NO:25; 5′-GCC AAC AAAA GGT CAG GTT G-3′, that allows the annealing and subsequent ligation to form a closed nucleic acid structure for further rounds of testing.

After various number of cycles are complete, (such as, for example, between about 5-10) the resultant 93 base pair PCR fragments are then closed and sequenced to reveal the preferred catalytic nucleic acid sequence that was selected for by doing these multiple rounds of trans-esterification reactions.

### Example 5

### Generation of a Mutation in a Target Sequence

In this embodiment, the targeting of green fluorescent protein (GFP) was carried out by the following procedure. A mutated form of green fluorescent protein was constructed by using the pEGFP-C1 (Clontech) and mutating the codon #40, which was changed from a tyrosine to a “stop” codon. This plasmid was then stably transfected into cell lines U87-MG (a human glioma cell line) and kidney 293 cells. The stably transfected cell lines were selected by the presence of G418 (neomycin) as positive selection pressure. Individual cell line clones that were resistant to G418 were sub-cloned and the presence of the stably integrated plasmid was detected by PCR amplification of the plasmid after isolating genomic DNA.

The assay scores positive cells that reverse the “stop” codon such that the cells will be able to fluoresce green. The phospho-imidazole-GFP AE-DNA oligonucleotide (an example of which is 5′-Imidazolide-Phosphate-CTTACG GCT AGC TGA CCG TTG GCT GTT CAT AA-(Catalytic N₁₃₋₁₅ sequence)-GAG GGG GAG GGG GAG GGA GGA GG-3′; SEQ ID NO:29) was mixed with Fugene (Roche) and applied to monolayer cell cultures that had been freshly plated one day prior to the experiment. The concentration of the AE-DNA was added from 1 micomolar to 10 micromolar concentration. After a 2 hour exposure, the cells were then washed and fresh media containing 10% FBS was added and the plates were incubated at 37° C. in the presence of 5% CO₂. Assay for green fluorescent cells was counted by flow cytometry and normalized to the efficiency of transfection for each specific cell type.

### Example 6

### Generation of a Mutation in a Target Sequence for Therapy

In some embodiments of the present invention, a wild-type nondefective sequence is replaced with a homologous sequence comprising a desirable alteration of the homologous sequence. In one exemplary embodiment, a mutation (or multiple mutations) is generated in a nucleic acid sequence to generate an environment more favorable to a particular therapy in an individual. For example, an endogenous gene product is mutated to make an individual more tolerable to a chemotherapy.

This example describes specific embodiments for therapeutic applications of the methods and compositions described herein. A skilled artisan recognizes based on the teachings provided herein that the present invention may be applied in vitro, in vivo, ex vivo, and so forth.

Primitive hematopoietic progenitors can be obtained from numerous sources including umbilical cord blood, bone marrow, mobilized peripheral blood cells, and even fetal liver. These hematopoietic stem cells (HSC) are generally identified as CD34+. As used herein, hematopoietic cells include both precursors and mature cells of the erythroid, lymphoid, monocytoid (macrophage) and granulocytic lineages. These pluripotent HSCs are capable of reconstituting a sub-lethally irradiated recipient with all blood cell lineages, and it is these aspects of HSCs that make them ideally suited for gene therapy strategies that can modify or correct a defective gene. In fact, several therapeutic HSC gene therapy trials are currently in progress for single gene disorders, such as Gaucher disease (Karlsson et al., 1996), chronic Granulomatous disease, Hunter syndrome, and adenosine deaminase deficiency (ADA). The preferred procedure would be to harvest preferably bone marrow HSCs in an affected individual, although other means of obtaining pluripotent cells are known in the art. In specific embodiments, the affected individual is pre-treated with pre-cytotoxic treatment and/or systemic primine with granulocyte colony stimulant factor (G-CSF), which tends to enrich the population of HSCs for harvesting.

Standard flow cytometry allows for the convenient method of isolation of CD34+cells. Culturing of the harvested cells in serum free media containing interleukin-3 (IL-3), interleukin-6 (IL-6), along with G-CSF, as well as stem cell factor and flt-3 ligand for brief periods of time (such as, for example, 6-12 hours) is sufficient, as well as culturing the CD34+cells on culture plates coated with the COOH-terminal fragment of fibronectin. After the brief culturing of HSCs under these conditions, generally improved abilities for transfection occur, as a larger portion of these cells begin cell cycle entry. At this time, oligonucleotides as described herein (which may represent various of the AE-DNA described herein or others generated based on teachings provided herein) can be introduced to effect genetic change. The preferred AE-DNA as described elsewhere herein would have already been activated by the addition of the 5′ end of the molecule with phospho-imidazolide. The conditioned CD34+HSCs are then incubated between 1 to 40 micromolar concentration of the AE-DNA alone or with a lipid carrier such as Lipofectin (Roche) or Fugene-6 transfection reagent (Roche) for about two to six hours. The cells are then washed prior to in vivo injection through intraperitoneal route (i.p.) or intravenous route (i.v.), as preferable. Alternatively, the activated oligonucleotides (AE-DNA) is introduced to the prepared cells by first gentle trypsinizing the inherent cells, washing them two times in PBS and re-suspending them in medium without serum. The cell mixture could then be electroporated by standard techniques with between about 1 to 40 micromolar concentration of the AE-DNA.

In some embodiments, the engraftment and expression of corrected gene product can simultaneously be injected into an immuno-compromised test animal, such as bnx/hu xenograft animals as well as beige/nude/bnx mice. These animals can then be used as recipients after sublethal radiation, which allows for simultaneous evaluation of the percentage of corrected cell population as well as the ability to test for full reconstitution of all hemopoietic cellular lineages. Finally the durability of the corrected genetic change can also be evaluated in these parallel test animals that received a fraction of the genetically modified HSCs.

In one specific and exemplary embodiment, an AE-DNA was designed to place a point mutation in the human Dihydrofolate Reductase (DHFR) nucleic acid sequence. This mutation would increase the resistance to the chemotherapy Methotrexate. In another specific and exemplary embodiment, an AE-DNA exchanges a defective human beta hemoglobin nucleic acid sequence.

In a specific embodiment, a patient's bone marrow stem cells are treated with the mutated DHFR composition, thus creating this mutation in the DHFR gene, and as a result the patient could then withstand much higher levels of Methotrexate (bone marrow protection strategy) for better tumor treatment.

Exemplary sequences for a DHFR-specific reaction include:

5′-Imidazo-Phospho-ACT CCC AAA GAA TGC GTTT CGC TGT CTC CGA TTG GA-(CGG AGC ATC AGT CTA)-TCC GAA GGg GAG UAA GGA U-3′ (SEQ ID NO:13);

Name: Uni-Enz-DHFR-10

5′-Phos-TGG AAG TAC TCC CAAAGA ATG GGA TTC TTT GGA AGC ATT CC TAC (AAC CAG TCG GAG AGG) AAG GA CTC GCC TCC GGT CCC A TCC-3′ (SEQ ID NO:14)

Name: Uni-Enz-DHFR-02

5′-Phos-TGG AAG TAC TCC CAAAGA ATG GGA TTC TTT GGA AGC ATT CC TAC (TGG TTG GTA AAA ATT)AAG GA CTC GCC TCC GGT CCC A TCC-3′ (SEQ ID NO:15)

In specific embodiments, the 5′ of the oligonucleotides are phosphorylated. In additional specific embodiments, there are phosphothioate linkages to the last 4 bases at the 3′ end of the oligos.

Deoxyoligonucleotides were synthesized, such as by automated phosphoramidite chemistry and then purified to homogeneity by denaturing PAGE and recovered from gel slices via the “modified crush and soak” method (Chen and Ruffner, 1986). These deoxyoligonucleotides were then purified again by HPLC with acetnitrile/water (at 1:1) and separated on C18 Sep-Pak reverse phase columns (Millipore). This was then followed by spin evaporation to dryness.

The formulation for synthetic oligonucleotides used for ex vivo treatment of hematopoetic stem cells (HSC) would allow for 80% of full “N-product length”, 10-15% of “(N−1)-product length”, and 5-10% of increasingly rare “(N−a)-products (wherein “a” represents increasing integers 2, 3, 4, 5, etc.). Prior to use, the deoxyoligonucleotides were quantitated spectrophotometrically at absorbance 260 nm. The deoxyoligonucleotides were then diluted to desired concentrations using Milli-Q-purified water. The sequence for each deoxyoligonucleotide is given for the preferred example for both the correction of dihydrofolate reductase gene sequence (DHFR) and for the correction of the mutated form of human beta-hemoglobin, the form of which is mutated at position 7 from the A:T to T:A transversion, which results in the sickling form of beta-hemoglobin (Hemoglobin-S) that is responsible for the disease entity of sickle cell anemia. Sequences provided herein allow for components of the AE-DNA to effectively cause, in this preferred case, a transesterification reaction with the resultant covalent attachment of the specific AE-DNA into one of the strands of the duplex DNA targets (in this exemplary case either to DHFR gene sequence or to the hemoglobin-S gene sequence).

The procedures outlined here can be applied to the sequences listed herein for the correction of human beta hemoglobin that contains the A:T transversion resulting in the sickle cell anemia phenotype. The A:T transversion at codon 7 causes a substitution of the amino acid valine for glutamic acid. This single amino acid change results in the hemoglobin-S form of the beta-globin chain and thereby has a tendency to polymerize in low oxygen tension environments into long fibers. These long fibers distort the shape of normal red blood cells making them stiff and sickled-shape leading to microvascular occlusion and stroke of multiple organs. The technique outline above with the transduction of HSC would be performed using an AE-DNA that is specifically designed to bind to and catalytically cause a transesterification reaction between the AE-DNA and the hemoglobin-S gene at the site of A:T transversion with the intent to restore the natural sequence and function of the gene. In one embodiment, an oligonucleotide, referred to as AE-DNA-Sickle-cell-open-cat, is utilized and is also referred to as 5′-Imidazo-Phospho-TCT CCA CAG GAG TCA CG TTT CGT CTA TCT GA CCG-(N₁₃₋₁₅)-CGG UAG AUG GUA AgG AUG GGA UAU-3′ (SEQ ID NO:21), wherein the N13-15 refers to a catalytic domain comprising 13-15 nucleotides, although this size may be greater or fewer.

The presence of “U” within the TFO portion is 5-Fluoro-deoxyuracil, which binds to the imperfect homopurine strand C:G inversions with strong affinity. Also, this AE-DNA comprises the general sequence used against the codon 7 correction scheme for hemoglobin-S but, as mentioned, does not specify a catalytic sequence. Many different catalytic sequences could be substituted in that region. This example of using alternate bases within the TFO portion reflects the flexibility of this molecule being a DNA molecule, an RNA molecule, or a DNA/RNA hybrid molecule. The presence of the “g” represents a T:A inversion “bulge” in the TFO binding region. A particular embodiment of a specific binding composition of the AE-DNA for sickle cell, including a specific enzymatic domain sequence is referred to as “AE-DNA Sickle cell-78-cat” 5′-Imidazo-Phospho-TCT CCA CAG GAG TCA CG TTT CGT CTA TCT GA CCG-(CGG AGC ATC AGT CTA)-CGG UAG AUG GUA AgG AUG GGA UAU-3′ (SEQ ID NO:22).

This overall technique would also be applicable to the AE-DNA oligonucleotide construct that is designed to cause a point mutation within the dihydrofolate reductase (DHFR) gene, such as the oligonucleotide referred to as AE-DNA-DHFR-open-cat, also referred to as SEQ ID NO:23 (5′-Imidazo-Phospho-ACT CCC AAA GAA TGC GTTT CGC TGT CTC CGA TTG GA-(N₁₃₋₁₅)-TCC GAA GGG GAG UAA GGA U-3′), wherein N is any nucleotide. The presence of “U” within the TFO portion is 5-Fluoro-deoxyuracil, which binds to the imperfect homopurine strand C:G inversions with strong affinity. Also, this AE-DNA comprises the general sequence used against the codon 35 (phenylalanine to serine) mutation scheme for Dihydrofolate Reductase but does not specify a catalytic sequence. Many different catalytic sequences could be substituted in that region, and sizes fewer than 13 nucleotides or greater than 15 nucleotides are applicable. The presence of the “g” represents a T:A inversion “bulge” in the TFO binding region.

As a specific exemplary embodiment, the oligonucleotide referred to as “AE-DNA-enzyme-DHFR-78” (SEQ ID NO:13) comprises a specific catalytic domain. This specific genetic alteration causes a change at codon position 22 from an amino acid leucine to tyrosine. This is brought about by a pyrimidine transition from C to T. Similar phenotypic changes in the dihydrofolate reductase gene can also be accomplished by changing codon 31 from a phenylalanine to a serine at position 31 and/or a mutation of leucine at position 22 can be changed to arginine. These point mutations that result in specific amino acid changes within the DHFR gene confer decreased binding affinity to the commonly used chemotherapeutic agent Methotrexate. Cells that contain one or more of these specific mutations allow significantly reduced sensitivity to Methotrexate. HSC's that have been modified to contain these mutations within the DHFR gene can spare a recipient animal from the hematological toxicity of Methotrexate, once engrafted. This could then be used as a strategy to allow treatment with higher dose Methotrexate to individuals receiving chemotherapy with less potential toxicity. These changes previously mentioned are specific for the murine form of the dihydrofolate reductase gene. A specific AE-DNA was constructed to target specifically the codon 35, which represents a phenylalanine to serine amino acid change. This represents a T to C change.

### REFERENCES

All patents and publications mentioned in the specification are indicative of the levels of those skilled in the art to which the invention pertains. All patents and publications are herein incorporated by reference to the same extent as if each individual publication was specifically and individually indicated to be incorporated by reference.

### PATENTS

U.S. Pat. No. 5,807,718

U.S. Pat. No. 5,861,288

U.S. Pat. No. 5,910,408

U.S. Pat. No. 6,110,462

U.S. Pat. No. 6,159,714

U.S. Pat. No. 6,326,174

PCT Patent Application WO 95/11304

EPO 0273085

### PUBLICATIONS

Beal, P A, Dervan, P B, 1991, Science, 251:1360-1363.

Benvenisty N, Reshef L. Proc Natl Acad Sci USA. 1986 December;83(24):9551-5.

Breaker et al., Chem Biol., 223-220, 1994.

Carmi et al., “Cleaving DNA with DNA,” Proc. Natl. Acad. Sci. USA, 95:2233-2237, 1998.

Cech, Curr. Opin. Struct. Biol., 2:605-609, 1992.

Chen and Okayama, Mol. Cell Biol., 7:2745-2752, 1987.

Chen, Z. and Ruffner, D. E. 1996 BioTechniques 21, 820-822.

Cuenoud et al., “DNA metalloenzyme with DNA ligase activity,” Nature, 375:611-614, 1995.

Dubensky T W, Campbell B A, Villarreal L P. Proc Natl Acad Sci USA. 1984 December;81(23):7529-33.

Durland, R H, et al, 1994, Nucleic Acids Research, 22:3233-3240.

Fechheimer et al., Proc. Natl. Acad. Sci. USA, 84:8463-8467, 1987.

Ferkol et al., FASEB J, 7:1081-1091, 1993.

Fischer, “Gene therapy of severe combined immunodeficiency. Interview with Alan Fischer,” J. Gene Med., 2(4):297-300, 2000.

Fraley et al., Proc. Natl. Acad. Sci. USA, 76:3348-3352, 1979.

Giovannangeli C, Helene C. Triplex-forming molecules for modulation of DNA information processing. Curr Opin Mol Ther 2000 June;2(3):288-96.

Gopal, Mol. Cell Biol., 5:1188-1190, 1985.

Grimm G N, Boutorine A S, Lincoln P, Helene C. Design and simple routes of synthesis of oligonucleotide conjugates for studies of DNA triple helix formation. Nucleosides Nucleotides Nucleic Acids 2001 April-July;20(4-7):909-14.

Hartigan-O'Connor and Chaberlain, “Developments in gene therapy for muscular dystrophy,” Microscopy Research & Technique, 48(3-4):223-238, 2000.

Hyde et al., “Repeat administration of DNA/liposomes to the nasal epithelium of patients with cystic fibrosis,” Gene Therapy, 7(13):1156-1165, 2000.

Igoucheva and Yoon, “Targeted single-base correction by RNA-DNA oligonucleotides,” Hum. Gene Ther., 11(16):2307-2312, 2000.

Ghosh and Bachhawat, In: Liver diseases, targeted diagnosis and therapy using specific receptors and ligands, Wu and Wu (Ed.), NY, Marcel Dekker, 87-104, 1991.

Graham and van der Eb, Virology, 52:456-467, 1973.

Kanavarioti, A.; Bernasconi, C. F., Doodokyan, D. L., and Alberas, D. L. J. Am. Chem. Soc. 111:7247-7257 (1989).

Kaneda et al., Science, 243:375-378, 1989.

Karlsson, S, et al, 1996, Genetic Therapy, 7:231-253.

Kato et al, J. Biol. Chem., 266:3361-3364, 1991.

Klein et al., Nature, 327:70-73, 1987.

Kohn, “Gene therapy for XSCID: the first success of gene therapy,” Pediatric Res., 48(5):578, 2000.

Kren et al., “Gene repair using chimeric RNA/DNA oligonucleotides,” Seminars in Liver Disease, 19(1):93-104, 1999.

Kren et al., “In vivo site-directed mutagenesis of the factor IX gene by chimeric RNA/DNA oligonucleotides,” See comments in Nat. Med., 4(3):274-275, 1998, Nat. Med., 4(3):285-290, 1998.

Li. et al., Proc. Natl. Acad. Sci. USA, 96:2746-2751, 1999.

Li J S, Fan Y H, Zhang Y, Marky L A, Gold B. J. Design of triple helix forming C-glycoside molecules. Am. Chem. Soc. Feb. 26, 2003;125(8):2084-93.

May et al., “Therapeutic haemoglobin synthesis in a beta-thalassaemic mice expressing lentivirus-encoded human beta-globin,” Nature, 406(6791):82-86, 2000.

Nicolau and Sene, Biochim. Biophys. Acta, 721:185-190, 1982.

Nicolau et al., Methods Enzymol., 149:157-176, 1987.

Olivas, W M, Maher, L J R 1995, Nucleic Acids Research, 23:1936-1941.

Perales et al., Proc. Nat'l Acad. Sci. USA, 91:4086-4090, 1994.

Potter et al., Proc. Nat'l Acad. Sci. USA, 81:7161-7165, 1984.

Rando et al., “Rescue of dystrophin expression in mdx mouse muscle by RNA/DNA oligonucleotides,” Proc. Natl. Acad. Sci. USA, 97(10):5363-5368, 2000.

Rippe et al., Mol. Cell Biol., 10:689-695, 1990.

Rohatgi, R., Bartel, D. P. and Szostak, J. W. J. Am. Chem. Soc. 118:3340-3344 (1996).

Rubenstein et al., “CFTR gene transduction in neonatal rabbits using an adeno-associated virus (AAV) vector,” Gene Therapy, 4(5):384-392, 1997.

Santoro et al., “A general purpose RNA-cleaving enzyme,” Proc. Natl. Acad. Sci. USA, 94:4262-4266, 1997.

Santoro et al., Biochemistry, 37:13330-13342, 1998.

Schwartz and Leterrier, “The IVth workshop on Duchenne muscular dystrophy gene therapy,” J. Gene Med., 1(6):444-448, 1999.

Singer et al., “Targeted mutagenesis of DNA with alkylating RecA assisted oligonucleotides,” Nucl. Acids Res., 27(24):e38, 1999.

Tur-Kaspa et al., Mol. Cell Biol., 6:716-718, 1986.

Vasquez et al., “Specific mutations induced by triplex-forming oligonucleotides in mice,” Science, 290(5491):530-533, 2000.

Vasquez K M, Glazer P M. Triplex-forming oligonucleotides: principles and applications. Q Rev Biophys. 2002 February;35(1):89-107.

Wagner et al., Science, 260:1510-1513, 1990.

Wong et al., Gene, 10:87-94, 1980.

Wu and Wu, Biochemistry, 27:887-892, 1988.

Wu and Wu, Adv. Drug Delivery Rev., 12:159-167, 1993.

Wu and Wu, J. Biol. Chem., 262:4429-4432, 1987.

Xu et al., “Activation of human gamma-globin gene expression via triplex-forming oligonucleotide,” Gene, 242(1−2):219-228, 2000.

Yang et al., Proc. Nat'l Acad. Sci. USA, 87:9568-9572, 1990.

Zelenin A V, Alimov A A, Titomirov A V, Kazansky A V, Gorodetsky S I, Kolesnikov V A. FEBS Lett. Mar. 11, 1991;280(1):94-6.

Zhu et al., “Targeted manipulation of maize genes in vivo using chimeric RNA/DNA oligonucleotides,” See Comments in Proc. Natl. Acad. Sci. USA, 96(15):8321-8323, 1999, Proc. Natl. Acad. Sci. USA, 96(15):8768-8773, 1999.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, manufacture, composition of matter, means, methods and steps described in the specification. As one of ordinary skill in the art will readily appreciate from the disclosure of the present invention, processes, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A nucleic acid molecule having at least one enzymatic domain that provides both phosphodiesterase hydrolysis and phosphodiesterification functions, wherein said domain is obtainable by a process comprising:
a) identifying a target DNA molecule having a known target sequence;
b) obtaining a tester nucleic acid molecule for testing for phosphodiesterase hydrolysis and phosphodiesterification activity;
c) assaying whether at least a part of the tester nucleic acid molecule facilitates insertion of a sequence into the target sequence; and
d) preparing the DNA molecule having the enzymatic domain by producing the molecule comprising the phosphodiesterase hydrolysis and phosphodiesterification activity sequence identified in the tester.

2. The nucleic acid molecule of claim 1, wherein the assaying step is further defined as:
providing a double stranded target DNA region, said target region defined as an acceptor region;
providing a single stranded donor molecule;
providing the tester molecule; and
assaying for action of said donor molecule upon said acceptor region.

3. The nucleic acid molecule of claim 2, wherein said assaying for action of said donor molecule upon said acceptor region is further defined as assaying for replacement of at least a part of said acceptor region with at least a part of said donor molecule.

4. The nucleic acid molecule of claim 2, wherein the tester molecule and the donor molecule are the same molecule.

5. The nucleic acid molecule of claim 4, wherein at least a portion of the tester molecule is further defined as comprising a folded complementary anti-parallel configuration.

6. The nucleic acid molecule of claim 2, wherein the double stranded DNA region is comprised of two monofilament molecules and wherein one of the monofilament molecules is the tester molecule.

7. The nucleic acid molecule of claim 1, wherein said phosphodiesterase hydrolysis and phosphodiesterification functions occur in a one-step process.

8. The nucleic acid molecule of claim 4, wherein said tester molecule is further defined as comprising one or more of the following:
an activated ligatable end;
an enzymatic domain, wherein said domain comprises the phosphodiesterase hydrolysis and phosphodiesterification functions; and
a DNA sequence homologous to the target DNA region.

9. The nucleic acid molecule of claim 8, wherein the tester molecule further comprises a triplex forming oligonucleotide domain.

10. The nucleic acid molecule of claim 8, wherein the activated ligatable end is located at the 5′ end of the DNA

11. The nucleic acid molecule of claim 8, wherein the activated ligatable end is located at the 3′ end of the DNA.

12. The nucleic acid molecule of claim 8, wherein the activated ligatable end comprises an activating group.

13. The nucleic acid molecule of claim 8, wherein the activated ligatable end comprises a phosphate group.

14. The nucleic acid molecule of claim 12, wherein the activating group is an imidazolide.

15. The nucleic acid molecule of claim 14, wherein the imidazolide is 4,5-imidazoledicarboxylic acid or 2-imidazolidonethione.

16. The nucleic acid molecule of claim 1, further comprising a phosphate group source.

17. The nucleic acid molecule of claim 16, wherein the phosphate group source is a nucleotide.

18. The nucleic acid molecule of claim 17, wherein the nucleotide is adenosine triphosphate.

19. The nucleic acid molecule of claim 8, wherein the activated ligatable end comprises a phosphor-imidazolide group.

20. The nucleic acid molecule of claim 8, wherein the activated ligatable end comprises adenine deoxyribonucleoside, guanine deoxyribonucleoside, thymine deoxyribonucleoside, cytosine deoxyribonucleoside, inositol ribonucleoside, or uracil ribonucleoside.

21. The nucleic acid molecule of claim 8, wherein the enzymatic domain comprises SEQ ID NO:5, SEQ ID NO:6, SEQ ID NO:7, SEQ ID NO:8, SEQ ID NO:9, SEQ ID NO:10, or SEQ ID NO:20.

22. The nucleic acid molecule of claim 8, wherein the target DNA region comprises human genomic sequence.

23. The nucleic acid molecule of claim 22, wherein the DNA sequence homologous to the target DNA region is further defined as comprising a therapeutic alteration compared to said target DNA region.

24. The nucleic acid molecule of claim 1, wherein said nucleic acid molecule is in a composition that further comprises a divalent cation.

25. The nucleic acid molecule of claim 24, wherein said divalent cation is Ba²⁺, Sr²⁺, Ca²⁺, Mg²⁺, Mn²⁺, Fe²⁺, Co²⁺, Ni²⁺, Cu²⁺², Zn²⁺, Pb²⁺, Cd²⁺, or a mixture thereof.

26. The nucleic acid molecule of claim 1, wherein said enzymatic domain comprises SEQ ID NO:5.

27. The nucleic acid molecule of claim 1, wherein said enzymatic domain comprises SEQ ID NO:6.

28. The nucleic acid molecule of claim 1, wherein said enzymatic domain comprises SEQ ID NO:7.

29. The nucleic acid molecule of claim 1, wherein said enzymatic domain comprises SEQ ID NO:8.

30. The nucleic acid molecule of claim 1, wherein said enzymatic domain comprises SEQ ID NO:9.

31. The nucleic acid molecule of claim 1, wherein said enzymatic domain comprises SEQ ID NO:10.

32. The nucleic acid molecule of claim 1, wherein said enzymatic domain comprises SEQ ID NO:20.

33. The nucleic acid molecule of claim 6, wherein the tester molecule is further defined as comprising the enzymatic domain, wherein said domain comprises the phosphodiesterase hydrolysis and phosphodiesterification functions.

34. The nucleic acid molecule of claim 6, wherein the tester molecule is a closed circular molecule.

35. The nucleic acid molecule of claim 2, wherein the assay step for action of said donor molecule upon said acceptor region comprises polymerase chain reaction.

36. A method of exchanging a nucleic acid sequence of interest with a target DNA region, comprising:
providing a nucleic acid molecule in accordance with claim 1, wherein said sequence inserted into the target sequence is referred to as the nucleic acid sequence of interest;
providing the target DNA region; and
introducing the nucleic acid sequence of interest to the target DNA region, wherein the phosphodiesterase hydrolysis and phosphodiesterification functions of the nucleic acid molecule from claim 1 exchanges at least a portion of said nucleic acid sequence of interest with said target DNA region.

37. The method of claim 36, wherein said nucleic acid molecule from claim 1 is further defined as comprising at least one of the following:
an activated ligatable end;
the enzymatic domain, said domain comprising the phosphodiesterase hydrolysis and phosphodiesterification functions; and
the nucleic acid sequence of interest, wherein said sequence is homologous to the target DNA region, wherein there is at least one nonidentical base pair between said nucleic acid sequence of interest and said target DNA region.

38. The method of claim 36, wherein the nucleic acid molecule from claim 1 further comprises a triplex forming oligonucleotide domain.

39. The method of claim 36, wherein said phosphodiesterase hydrolysis and/or said phosphodiesterification functions further comprise the use of histidine.

40. The method of claim 36, wherein said phosphodiesterase hydrolysis and/or said phosphodiesterification functions further comprise the use of a divalent cation.

41. The method of. Claim 36, wherein said method occurs under physiological conditions.

42. The method of claim 36, wherein said method occurs in vitro.

43. The method of claim 36, wherein said method occurs in vivo.

44. The method of claim 43, wherein said method occurs in a cell.

45. The method of claim 44, wherein said cell is in a human afflicted with a disease of genetic origin, said disease the indirect or direct result of a defect in said target DNA region.

46. A method of treating an individual afflicted with a disease of genetic origin, said disease of genetic origin comprising a defective DNA sequence, comprising the step of exchanging a nondefective DNA sequence with the defective DNA sequence using a DNA prepared in accordance with claim 1.

47. The method of claim 46, wherein said defect in said DNA sequence is a point mutation, an inversion, a deletion, a frameshift mutation, or a combination thereof.

48. The method of claim 46, wherein said defect in said DNA sequence comprises an error in a splicing mechanism or an error in a regulatory mechanism.

49. A method of treating an individual afflicted with a disease related to an undesirable gene product by affecting the gene product level or activity in a cell of the individual, said method comprising the step of exchanging a first DNA sequence with a second DNA sequence using a DNA prepared in accordance with claim 1, and wherein said exchanging step results in said affecting the gene product level or activity.

50. The method of claim 49, wherein said affecting the gene product level or activity comprises:
introducing a stop codon into nucleotide sequence that encodes the undesirable gene product;
reducing the transcriptional level or rate of the undesirable gene product;
altering post-transcriptional processing of the undesirable gene product;
or a combination thereof.

51. The method of claim 49, wherein the catalysis function for said phosphodiesterase hydrolysis and phosphodiesterification reactions is provided by a DNA molecule comprising the first DNA sequence.

52. A method of identifying a nucleic acid molecule comprising at least one enzymatic domain that provides both phosphodiesterase hydrolysis and phosphodiesterification functions, comprising:
a) identifying a target DNA molecule having a known target sequence;
b) obtaining a tester nucleic acid molecule for testing for the desired enzymatic activity;
c) assaying whether at least a part of the tester nucleic acid molecule facilitates insertion of a sequence into the target sequence; and
d) preparing the DNA molecule having the enzymatic domain by producing the molecule comprising the enzymatic activity sequence identified in the tester.

53. A nucleic acid molecule comprising an enzymatic DNA that provides both phosphodiesterase hydrolysis and phosphodiesterification functions identified by the method of claim 52.
